(19) United States
(12) Patent Application Publication
(10) Pub. No.: US 2025/0115938 A1
(43) Pub. Date: Apr. 10, 2025

(54) METHOD FOR PRODUCING GUANIDINO ACETIC ACID (GAA)

(71) Applicant: Evonik Operations GmbH, Essen (DE)

(72) Inventors: Frank Schneider, Halle (DE); Kay Marin, Borgholzhausen (DE); Melanie Nickolaus, Spenge (DE); Julia Tegethoff, Halle (Westf.) (DE); Marleen Oesterhoff, Paderborn (DE)

(73) Assignee: Evonik Operations GmbH, Essen (DE)

(21) Appl. No.: 18/870,088

(22) PCT Filed: May 24, 2023
(86) PCT No.: PCT/EP2023/063902

§ 371 Date: Nov. 27, 2024

(30) Foreign Application Priority Data
Jun. 3, 2022 (EP) 22177256.9

(51) Int. Cl. C12P 13/04; C12N 9/06; C12N 9/10; C12N 9/12; C12N 15/77; C12P 13/10

(52) U.S. Cl. CPC C12P 13/04; C12N 9/0016; C12N 9/1007; C12N 9/1096; C12N 9/1217; C12N 15/77; C12P 13/10; C12Y 201/01002; C12Y 201/04001; C12Y 206/01044; C12Y 207/02002

(57) ABSTRACT

A microorganism is capable of producing guanidinoacetic acid (GAA) that has been improved by using a carbamate kinase. The microorganism with an improved capacity to provide L-arginine as starting material of the GAA biosynthesis by efficient recycling of ornithine improves the production process of GAA. A method for the fermentative production of GAA and a method for the fermentative production of creatine include the incorporation of the microorganism.

Guanidino acetic acid (GAA) is a colorless crystalline organic compound used as animal feed additive (e.g. WO 2005120246 A1 and US2011257075 A1). GAA is a natural precursor of creatine (e.g. Humm et al., Biochem. J. (1997) 322, 771-776). Therefore, the supplementation of GAA allows for an optimal supply of creatine in the organism.

The present invention pertains to a microorganism transformed to be capable of producing guanidinoacetic acid (GAA) and to a method for the fermentative production of GAA using such microorganism. The present invention also relates to a method for the fermentative production of creatine.

In biological systems GAA and ornithine are formed from arginine and glycine as starting materials by the catalytic action of an L-arginine:glycine-amidinotransferase (AGAT; EC 2.1.4.1). This reaction is also the first step in creatine biosynthesis.

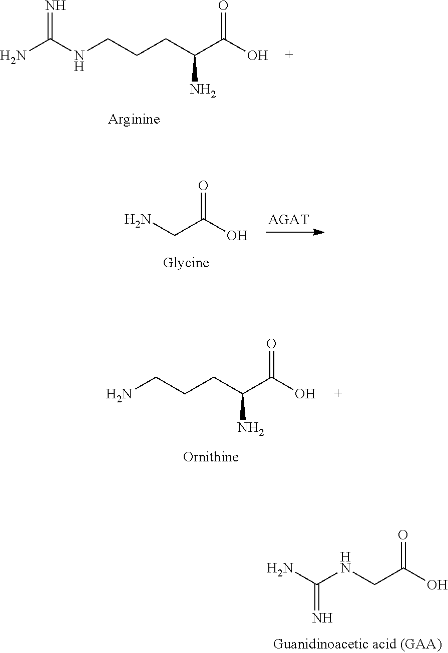

Guthmiller et al. (J Biol Chem. 1994 Jul. 1; 269 (26): 17556-60) have characterized a rat kidney AGAT by cloning and heterologously expressing the enzyme in Escherichia coli (E. coli). Muenchhoff et al. (FEBS Journal 277 (2010) 3844-3860) report the first characterization of an AGAT from a prokaryote also by cloning and heterologously expressing the enzyme in E. coli.

Fan Wenchao discloses a method for the production of creatine by fermentation of non-pathogenic microorganisms, such as Corynebacterium glutamicum (CN 106065411 A). The microorganism has the following biotransformation functions: glucose conversion to L-glutamic acid; conversion of L-glutamic acid to N-acetyl-L-glutamic acid; conversion of N-acetyl-L-glutamic acid to N-acetyl-L-glutamic acid semialdehyde; conversion of N-acetyl-L-glutamic acid semialdehyde to N-acetyl-L-ornithine; conversion of N-acetyl-L-ornithine to L-ornithine; conversion of L-ornithine to L-citrulline; conversion of L-citrulline to arginino-succinic acid; conversion of arginino-succinic acid to L-arginine; conversion of L-arginine to guanidinoacetic acid; and, finally, conversion of guanidinoacetic acid to creatine. Fan Wenchao proposes, that the microorganism overexpresses one or more enzymes selected from the group consisting of N-acetylglutamate-synthase, N-acetylornithine-o-aminotransferase, N-acetylornithinase, ornithine-carbamoyl transferase, argininosuccinate synthetase, glycine amidino-transferase (EC: 2.1. 4.1), and guanidinoacetate N-methyltransferase (EC: 2.1.1.2). The microorganism overexpresses preferably glycine aminotransferase (L-arginine:glycine amidinotransferase) and guanidinoacetate N-methyltransferase.

A microorganism capable of producing guanidinoacetic acid (GAA) was published by Zhang et al. (ACS Synth. Biol. 2020, 9, 2066-275). They designed a reconstituted the ornithine cycle in E. coli by introducing a heterologous AGAT from different species (e.g., Homo sapiens, Cylindrospermopsis raciborskii, Moorea producens) and by introducing a citrulline synthesis module (e.g. ovexpression of carAB, argF and argl) and an arginine synthesis module (e.g. overexpression of argG, argH; introduction of aspA) into E. coli.

Schneider and Jankowitsch (WO 2021122400 A1) propose a method to produce GAA using a microorganism having gene coding for a protein having the function of an L-arginine:glycine amidinotransferase and an increased carbamolyphosphate synthase. The carbamoyl phosphate is an important precursor for the biosynthesis of GAA but also for L-arginine and other compounds.

Several approaches for increasing the production of one of the starting materials in GAA synthesis, i.e. L-arginine, in microorganisms, particularly bacteria, are also known from literature. An overview for the metabolic engineering of Corynebacterium glutamicum (C. glutamicum) for L-arginine production is provided by Park et al. (NATURE COMMUNICATIONS|DOI: 10.1038/ncomms5618). Yim et al. (J Ind Microbiol Biotechnol (2011) 38:1911-1920) could show that inactivation of the argR, gene coding for the central repressor protein ArgR controlling the L-arginine biosynthetic pathway, by disrupting the chromosomal argR gene in C. glutamicum leads to an improved arginine-producing strain. Ginesy et al. (Microbial Cell Factories (2015) 14:29) report the successful engineering of E. coli for enhanced arginine production. Among other, they proposed the deletion of the argR repressor gene.

Wang et al. (Applied Microbiology and Biotechnology, 2021, vol. 105, pp. 3265-3276; https://doi.org/10.1007/s00253-021-11242-w) underlined that carbamoyl phosphate is essential for L-arginine production also in Corynebacterium sp. They showed among other that the overexpression of the carAB gene encoding a carbamoyl phosphate synthetase and the introduction of a heterologous gene (from Enterococcus faecalis) coding for a carbamate kinase (CK) which catalyzes synthesis of carbamoyl phosphate from inorganic ammonia, hydrogencarbonate and ATP can lead to an increase of L-arginine production. The advantage of using a carbamate kinase results from the utilization of inorganic ammonium as nitrogen source by this enzyme. In comparison to the carbamoyl phosphate synthetase, using glutamine as nitrogen source, the carbamate kinase allows a reduction of the overall energy demand for the formation of carbamoyl phosphate. Yan et al. (Fermentation 2022, vol. 8, no. 3, 7 Mar. 2022, p. 116; doi: 10.3390/fermentation8030116) disclose the biosynthesis of GAA by a whole-cell catalysis with Bacillus subtilis by introducing a heterologous AGAT gene into B. subtilis, optimizing the expression level of the AGAT gene, optimizing the natural ornithine cycle and knocking-out the first gene of the glycine degradation pathway, the glycine dehydrogenase gene gcvP.

Schneider and Jankowitsch (WO 2022008276 A1) propose to produce GAA using recombinant microorganisms comprising a gene coding for a L-arginine:glycine amidinotransferase (AGAT) and, in order to increase the production of one of the starting materials, glycine, a reduced or deleted malate synthase gene and optionally an overexpressed gene coding for a glyoxylate aminotransferase. They also disclose that the carbamate kinase (CK) may contribute to arginine production.

To increase the production of GAA using a microorganism an intracellular high amount of the starting materials arginine and/or glycine are necessary. At the same time the byproduct of the AGAT reaction, ornithine, has to be recycled to arginine efficiently in order to prevent loss of carbon and energy.

The problem underlying the present inventions is to provide a microorganism transformed to be capable for producing guanidinoacetic acid (GAA), in particular a microorganism with an improved capacity to provide L-arginine as starting material of the GAA biosynthesis by efficient recycling of ornithine, and a method for the fermentative production of GAA using such microorganism.

The problem is solved by a microorganism comprising at least one heterologous gene coding for a protein having the function of a L-arginine:glycine amidinotransferase (AGAT, e.g. EC2.1.4.1) and comprising at least one gene coding for a protein having the function of a carbamate kinase (CK, e.g. EC 2.7.2.2) and further comprising at least one gene coding for a protein having the function of a NADH-dependent amino acid dehydrogenase.

A heterologous gene means that the gene has been inserted into a host organism which does not naturally have this gene. Insertion of the heterologous gene in the host is performed by recombinant DNA technology. Microorganisms that have undergone recombinant DNA technology are called transgenic, genetically modified or recombinant. A heterologous protein means a protein that is not naturally occurring in the microorganism. A homologous or endogenous gene means that the gene including its function as such or the nucleotide sequence of the gene is naturally occurring in the microorganism or is “native” in the microorganism. A homologous or a native protein means a protein that is naturally occurring in the microorganism.

In the microorganism according to the present invention the protein having the function of an L-arginine:glycine amidinotransferase (AGAT) comprises an amino acid sequence which is at least 80% identical to the amino acid sequence according to SEQ ID NO:9.

In the microorganism according to the present invention the at least one gene coding for a protein having the function of a carbamate kinase (CK, e.g. EC 2.7.2.2) may be heterologous.

In the microorganism according to the present invention the activity of the least one protein having the function of a carbamate kinase is increased compared with the respective activity in the wildtype microorganism.

Generally, increased enzyme activities in the microorganism can be achieved, for example, by mutation of the corresponding endogenous gene. A further measure to increase enzymatic activities may be to stabilize the mRNA coding for the enzymes. Increased enzyme activities in the microorganism may also be achieved by overexpression of the genes coding for the respective enzymes.

The microorganism according to the present invention may comprise at least one heterologous gene coding for a protein having the function of a carbamate kinase. In the microorganism of the present invention the at least one protein having the enzymic activity of a carbamate kinase may comprise an amino acid sequence which is at least 80% identical to the amino acid sequence according to SEQ ID NO:6.

Overexpression of a gene is generally achieved by increasing the copy number of the gene and/or by functionally linking the gene with a strong promoter and/or by enhancing the ribosomal binding site and/or by codon usage optimization of the start codon or of the whole gene or a combination comprising a selection of all methods mentioned above.

A promoter is a DNA sequence consisting of about 40 to 50 base pairs and which constitutes the binding site for an RNA polymerase holoenzyme and the transcriptional start point, whereby the strength of expression of the controlled polynucleotide or gene can be influenced. Generally, it is possible to achieve an overexpression or an increase in the expression of genes in bacteria by selecting strong promoters, for example by replacing the original promoter with strong, native (originally assigned to other genes) promoters or by modifying certain regions of a given, native promoter (for example its so-called -10 and -35 regions) towards a consensus sequence, e.g. as taught by M. Patek et al. (Microbial Biotechnology 6 (2013), 103-117) for C. glutamicum. An example for a “strong” promoter is the superoxide dismutase (sod) promoter (“Psod”; Z. Wang et al., Eng. Life Sci. 2015, 15, 73-82). A “functional linkage” is understood to mean the sequential arrangement of a promoter with a gene, which leads to a transcription of the gene.

In a particular embodiment of the present invention the gene coding for a protein having the function of a carbamate kinase is functionally linked to a strong promoter. Preferably, the promoter is the superoxide dismutase (sod) promoter (“Psod”).

In the microorganism according to the present invention the protein having the function of a NADH-dependent amino acid dehydrogenase may be a heterologous protein.

NADH depending amino acid dehydrogenases (AaDH) catalyse the amination reaction of a keto acid to L-amino acid; the NADH depending amino acid dehydrogenases are important for the assimilation or dissimilation of ammonium in a cell.

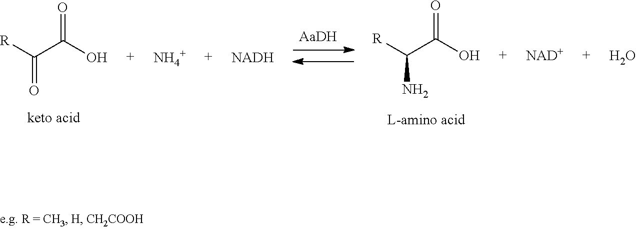

Since most of these amino acid dehydrogenases can use different a-keto acids as substrate, they are often annotated with different EC numbers. However, all these amino acid dehydrogenases have in common that they assimilate ammonium, or, in case of the reverse reaction, dissimilate ammonium.

Examples for different amino acid dehydrogenases are the following:

Reaction EC 1.4.1.1: Alanine dehydrogenase:

pyruvate+NH3+NADH+H+<->L-alanine+H2O+NAD+

Reaction EC 1.4.1.10: Glycine dehydrogenase:

glyoxylate+NH3+NADH+H+<->glycine+H2O+NAD+

Reaction EC 1.4.1.21: Aspartate dehydrogenase:

oxaloacetate+NH3+NADH+H+<->L-aspartate+H2O+NAD+

Several NADH depending amino acid dehydrogenase (AaDH) Proteins are known in literature which accept a broad range of substrates and can therefore often aminate several different keto acids to the corresponding amino acids. (Fernandes et al. Protein Engineering, Design & Selection, 2015, vol. 28 no. 2, pp. 29-35; Giffin et al., Journal of Bacteriology, 2012, vol. 194 no. 5, pp. 1045-1054; Phogosee et al., Archives of Microbiology, 2018 vol. 200 pp. 719-727; Schuffenhauer et al. 1999, vol. 171, pp 417-423; Vancura et al. Eur J Biochem 1989, vol. 179, pp. 221-227, Yoshida and Freese, Biochim. Biophys. Acta, 1965, vol. 96, pp 248-262)

Therefore, no clear link of the reaction to an EC number exists. Table 1 shows some examples:

TABLE 1
Examples for NADH depending amino acid dehydrogenase (AaDH) Proteins

| | AaDH | AaDH | | | |
|---|---|---|---|---|---|
| | Myco- | Myco- | AaDH | AaDH | AaDH |
| | bacterium | bacterium | Bacillus | Streptomyces | Aphanothece |
| Reaction | tuberculosis | smegmatis | subtilis | fradiae | halophytica |
| Pyruvat->L-Alanin | yes (1) | yes (3) | yes (4) | yes (5) | yes (6) |
| L-Alanin->Pyruvat | yes (1) | yes (3) | yes (4) | yes (5) | yes (6) |
| Glyoxylat->Glycin | yes (1) | yes (3) | yes (4) | no(5) | yes (6) |
| Oxalacetat-> | yes (1) | yes (3) | no data | yes (5) | no data |
| Aspartat | | | | | |
| Hydroxypyruvat -> | yes (1) | no data | yes (4) | no data | no data |
| Serin | | | | | |
| Methylglyoxal -> | yes (1) | no data | no data | no data | no data |
| Aminoaceton | | | | | |
| 4-Hydroxy-2- | yes (2) | no data | no data | no data | no data |
| oxobutyrat -> L- | | | | | |
| Homoserin | | | | | |
| 2-Oxobutyrate -> 2- | no data | yes (3) | yes (4) | yes(5) | no data |
| aminobutyrate | | | | | |
| Literature | (1) Giffin, 2012 | (3) | (4) Yoshida, | (5) Vancura, | (6) Phogosee, |
| | (2) Fernandes, | Schuffen- | 1965 | 1989 | 2019 |
| | 2015 | hauer, | | | |
| | | 1999 | | | |

In the microorganism according to the present invention the at least one protein having the function of a NADH-dependent amino acid dehydrogenase may be selected from the group consisting of alanine dehydrogenase (EC 1.4.1.1), glycine dehydrogenase (EC 1.4.1.10) and aspartate dehydrogenase (EC 1.4.1.21).

In the microorganism of the present invention the activity of the at least one NADH-dependent amino acid dehydrogenase may be increased compared with the respective activity in the wildtype microorganism.

The at least one protein having the function of a NADH-dependent amino acid dehydrogenase is preferably heterologous.

The at least one NADH-dependent amino acid dehydrogenase comprised in the microorganism according to the present invention may be selected from the group consisting of alanine dehydrogenase (EC 1.4.1.1), glycine dehydrogenase (EC 1.4.1.10) and aspartate dehydrogenase (EC 1.4.1.21).

In the microorganism of the present invention the protein having the function of a NADH-dependent amino acid dehydrogenase may comprise an amino acid sequence which is at least 80% identical to the amino acid sequence according to SEQ ID NO:13, according to SEQ ID NO:21, according to SEQ ID NO:22, according to SEQ ID NO:23 or according to SEQ ID NO:24.

The microorganism of the present invention may further comprise at least one gene coding for a protein having the function of a glyoxylate aminotransferase.

Several glyoxylate amino transferases are known and vary in their substrate specificity with respect to the amino donor (cf. e.g. Kameya et al. FEBS Journal 277 (2010) 1876-1885; Liepman and Olsen, Plant Physiol. Vol. 131, 2003, 215-227; Sakuraba et al., JOURNAL OF BACTERIOLOGY, August 2004, p. 5513-5518; Takada and Noguchi, Biochem. J. (1985) 231, 157-163). Since most of these glyoxylate aminotransferase can use different amino acids as amino donors, they are often annotated with different EC numbers. However, all these aminotransferases have in common that they use glyoxylate as acceptor molecule, or, in case of the reverse reaction, glycine as donor molecule. Examples for a protein having the function of a glyoxylate aminotransferase are the following:

Glycine transaminase (EC 2.6.1.4) catalyzes the reaction:

L-glutamate+glyoxylate<=>alpha-ketoglutarate+glycine.

Glycine: oxaloacetate transaminase (EC 2.6.1.35) catalyzes the reaction:

L-aspartate+glyoxylate<=>oxaloacetate+glycine.

Alanine: glyoxylate transaminase (EC 2.6.1.44) catalyzes the reaction:

L-alanine+glyoxylate<=>pyruvate+glycine.

Serine: glyoxylate transaminase (EC 2.6.1.45) catalyzes the reaction:

L-serine+glyoxylate<=>3-hydroxy-pyruvate+glycine.

Methionine: glyoxylate transaminase (EC 2.6.1.73) catalyzes the reaction:

L-methionine+glyoxylate<=>4-(methylsulfanyl)-2-keto-butanoate+glycine.

The aromatic amino acid: glyoxylate transaminase (EC 2.6.1.60) catalyzes the reaction:

aromatic amino acid+glyoxylate<=>aromatic keto-acid+glycine.

Kynurenine: glyoxylate transaminase (EC 2.6.1.63) catalyzes the reaction:

kynurenine+glyoxylate<=>4-(2-aminophenyl)-2,4-diketo-butanoate+glycine.

(S)-Ureido-glycine: glyoxylate transaminase (EC 2.6.1.112) catalyzes the reaction:

(S)-ureido-glycine+glyoxylate<=>N-carbamoyl-2-keto-glycine+glycine.

In a further embodiment of the present invention the enzymic activity of the at least one protein having the function of a glyoxylate aminotransferase is increased compared to the respective enzymic activity in the wildtype microorganism.

The at least one protein having the function of a glyoxylate aminotransferase is preferably heterologous.

In a particular embodiment of the present invention the at least one protein having the function of a glyoxylate aminotransferase is a glycin: glyoxylate aminotransferase.

In the microorganism of the present invention the protein having the enzymic activity of a glyoxylate aminotransferase may comprise an amino acid sequence which is at least 80% identical to the amino acid sequence according to SEQ ID NO: 16, according to SEQ ID NO: 19 or according to SEQ ID NO:20.

The microorganism according to the present invention may have an increased ability to produce L-arginine from L-ornithine compared with the ability of the wildtype microorganism.

In the context of the present invention, a microorganism having an increased ability to produce L-arginine means a microorganism producing L-arginine in excess of its own need. Examples for such L-arginine producing microorganisms are e.g. C. glutamicum ATCC 21831 or those disclosed by Park et al. (NATURE COMMUNICATIONS|DOI: 10.1038/ncomms5618) or by Ginesy et al. (Microbial Cell Factories (2015) 14:29). In contrast to formerly described microorganisms having an increased ability to produce L-arginine, L-arginine excretion is not necessary in strains for GAA production since arginine is utilized inside the cell in the framework of the present invention for GAA production.

In a further embodiment the microorganism according to the present invention the expression of an argR gene coding for the arginine responsive repressor protein ArgR is attenuated compared to the expression of the argR gene in the wildtype microorganism. Alternatively, the argR gene is deleted

The microorganism of the present invention may belong to the genus Corynebacterium, to the genus Bacillus (Yan, K., et al. (2022). “Biosynthesis of Guanidinoacetate by Bacillus subtilis Whole-Cell Catalysis.” Fermentation 8 (3): 116), to the genus Enterobacteriaceae or to the genus Pseudomonas.

In a particular embodiment of the present invention the microorganism is Corynebacterium glutamicum (C. glutamicum) or Escherichia coli (E. coli).

The present invention further concerns a method for the fermentative production of guanidino acetic acid (GAA), comprising the steps of a) cultivating the microorganism according to the present invention in a medium, and b) accumulating GAA in the medium to form a GAA containing fermentation broth.

Preferably, the method further comprises isolating GAA from the GAA containing fermentation broth.

In a particular embodiment the microorganism of the present invention further comprises a gene coding for an enzyme having the activity of a guanidinoacetate N-methyltransferase. The gene coding for an enzyme having the activity of a guanidinoacetate N-methyltransferase may be overexpressed.

The present invention also concerns a method for the fermentative production of creatine, comprising the steps of a) cultivating the microorganism according to the present invention further comprising a gene coding for an enzyme having the activity of a guanidinoacetate N-methyltransferase in a suitable medium under suitable conditions, and b) accumulating creatine in the medium to form a creatine containing fermentation broth.

Preferably, the method further comprises isolating creatine from the creatine containing fermentation broth. creatine may be extracted from fermentation broth by isoelectric point method and/or ion exchange method. Alternatively, creatine can be further purified by a method of recrystallization in water.

### EXPERIMENTAL SECTION

### A) Materials and Methods

### Chemicals

Kanamycin solution from Streptomyces kanamyceticus was purchased from Sigma Aldrich (St. Louis, USA, Cat. no. K0254). If not stated otherwise, all other chemicals were purchased analytically pure from Merck (Darmstadt, Germany), Sigma Aldrich (St. Louis, USA) or Carl-Roth (Karlsruhe, Germany).

### Cultivation for Cell Proliferation

If not stated otherwise, cultivation/incubation procedures were performed as follows herewith:
a. LB broth (MILLER) from Merck (Darmstadt, Germany; Cat. no. 110285) was used to cultivate E. coli strains in liquid medium. The liquid cultures (10 ml liquid medium per 100 ml Erlenmeyer flask with 3 baffles) were incubated in the Infors HT Multitron standard incubator shaker from Infors GmbH (Bottmingen, Switzerland) at 30° C. and 200 rpm.
b. LB agar (MILLER) from Merck (Darmstadt, Germany, Cat. no. 110283) was used for cultivation of E. coli strains on agar plates. The agar plates were incubated at 30° C. in an INCU-Line® mini incubator from VWR (Radnor, USA).
c. Brain heart infusion broth (BHI) from Merck (Darmstadt, Germany, Cat. no. 110493) was used to cultivate C. glutamicum strains in liquid medium. The liquid cultures (10 ml liquid medium per 100 ml Erlenmeyer flask with 3 baffles) were incubated in the Infors HT Multitron standard incubator shaker from Infors GmbH (Bottmingen, Switzerland) at 30° C. and 200 rpm.
d. Brain heart agar (BHI-agar) from Merck (Darmstadt, Germany, Cat. no. 113825) was used for cultivation of C. glutamicum strains on agar plates. The agar plates were incubated at 30° C. in an incubator from Heraeus Instruments with Kelvitron® temperature controller (Hanau, Germany).
e. For cultivating C. glutamicum after electroporation, BHI-agar (Merck, Darmstadt, Germany, Cat. no. 113825) was supplemented with 134 g/l sorbitol (Carl Roth GmbH+Co. KG, Karlsruhe, Germany), 2.5 g/l yeast extract (Oxoid/ThermoFisher Scientific, Waltham, USA, Cat. no. LP0021) and 25 mg/l kanamycin. The agar plates were incubated at 30° C. in an incubator from Heraeus Instruments with Kelvitron® temperature controller (Hanau, Germany).

### Determining Optical Density of Bacterial Suspensions

a. The optical density of bacterial suspensions in shake flask cultures was determined at 600 nm (OD600) using the Bio-Photometer from Eppendorf AG (Hamburg, Germany).
b. The optical density of bacterial suspensions produced in the Wouter Duetz (WDS) micro fermentation system (24-Well Plates) was determined at 660 nm (OD660) with the GENios™ plate reader from Tecan Group AG (Männedorf, Switzerland).

### Centrifugation

a. Bacterial suspensions with a maximum volume of 2 ml were centrifuged in 1.5 ml or 2 ml reaction tubes (e.g. Eppendorf Tubes® 3810X) using an Eppendorf 5417 R benchtop centrifuge (5 min. at 13.000 rpm).
b. Bacterial suspensions with a maximum volume of 50 ml were centrifuged in 15 ml or 50 ml centrifuge tubes (e.g. Falcon™ 50 ml Conical Centrifuge Tubes) using an Eppendorf 5810 R benchtop centrifuge for 10 min. at 4.000 rpm.

### DNA Isolation

Plasmid DNA from E. coli cells was isolated using the QIAprep Spin Miniprep Kit from Qiagen (Hilden, Germany, Cat. No. 27106) according to the instructions of the manufacturer.

### Polymerase Chain Reaction (PCR)

PCR with a proof reading (high fidelity) polymerase was used to amplify a desired segment of DNA for sequencing or DNA assembly cloning. Non-proof-reading polymerase Kits were used for determining the presence or absence of a desired DNA fragment directly from E. coli or C. glutamicum colonies.
a. The Phusion® High-Fidelity DNA Polymerase Kit (Phusion Kit) from New England BioLabs Inc. (Ipswich, USA, Cat. No. M0530) was used for template-correct amplification of selected DNA regions according to the instructions of the manufacturer (see Table 2).

TABLE 2
PCR Program

| | | T | |
|---|---|---|---|
| Step | Time [min.:sec.] | [° C.] | Description |
| 1 | 00:30 | 98 | Initial denaturation step |
| 2 | 00:05 | 98 | Denaturation step |
| 3 | 00:30 | 60 | Annealing step |
| 4 | 30 sec. per kb DNA | 72 | Elongation step |
| | | | Repeat step 2 to 4: 35 x |
| 5 | 05:00 | 72 | Final elongation step |
| 6 | Hold | 4 | Cooling step |

Thermocycling conditions for PCR with Phusion ® High-Fidelity DNA Polymerase Kit from New England BioLabs Inc.

b. Taq PCR Core Kit (Taq Kit) from Qiagen (Hilden, Germany, Cat. No. 201203) was used to amplify a desired segment of DNA to confirm its presence. The kit was used according to the instructions of the manufacturer (see Table 3).

TABLE 3
PCR Program

| | | T | |
|---|---|---|---|
| Step | Time [min.:sec.] | [° C.] | Description |
| 1 | 05:00 | 94 | Initial denaturation step |
| 2 | 00:30 | 94 | Denaturation step |
| 3 | 00:30 | 52 | Annealing step |
| 4 | 1 min. per kb DNA | 72 | Elongation step |
| | | | Repeat step 2 to 4: 35 x |
| 5 | 04:00 | 72 | Final elongation step |
| 6 | Hold | 4 | Cooling step |

Thermocycling conditions for PCR with Taq PCR Core Kit (Taq Kit) from Qiagen.

c. SapphireAmp® Fast PCR Master Mix (Sapphire Mix) from Takara Bio Inc (Takara Bio Europe S.A.S., Saint-Germain-en-Laye, France, Cat. No. RR350A/B) was used as an alternative to confirm the presence of a desired segment of DNA in cells taken from E. coli or C. glutamicum colonies according to the instructions of the manufacturer (see Table 4).

TABLE 4
PCR Program

| | | T | |
|---|---|---|---|
| Step | Time [min.:sec.] | [° C.] | Description |
| 1 | 01:00 | 94 | Initial denaturation step |
| 2 | 00:05 | 98 | Denaturation step |
| 3 | 00:05 | 55 | Annealing step |
| 4 | 10 sec. per kb DNA | 72 | Elongation step |
| | | | Repeat step 2 to 4: 30 x |
| 5 | 04:00 | 72 | Final elongation step |
| 6 | Hold | 4 | Cooling step |

Thermocycling conditions for PCR with SapphireAmp ® Fast PCR Master Mix (Sapphire Mix) from Takara Bio Inc.

d. All oligonucleotide primers were synthesized by Eurofins Genomics GmbH (Ebersberg, Germany).
e. As PCR template either a suitably diluted solution of isolated plasmid DNA or of total DNA isolated from a liquid culture or the total DNA contained in a bacterial colony (colony PCR) was used. For said colony PCR the template was prepared by taking cell material with a sterile toothpick from a colony on an agar plate and placing the cell material directly into the PCR reaction tube. The cell material was heated for 10 sec. with 800 W in a microwave oven type Mikrowave & Grill from SEVERIN Elektrogeräte GmbH (Sundern, Germany) and then the PCR reagents were added to the template in the PCR reaction tube.
f. All PCR reactions were carried out in PCR cyclers type Mastercycler or Mastercycler nexus gradient from Eppendorf AG (Hamburg, Germany).

### Restriction Enzyme Digestion of DNA

For restriction enzyme digestions either “FastDigest restriction endonucleases (FD)” (ThermoFisher Scientific, Waltham, USA) or restriction endonucleases from New England BioLabs Inc. (Ipswich, USA) were used. The reactions were carried out according to the instructions of the manufacturer's manual.

### Determining the Sizes of DNA Fragments

a. The sizes of small DNA fragments (<1000 bps) were usually determined by automatic capillary electrophoresis using the QIAxcel from Qiagen (Hilden, Germany).
b. If DNA fragments needed to be isolated or if the DNA fragments were >1000 bps DNA was separated by TAE agarose gel electrophoresis and stained with GelRed® Nucleic Acid Gel Stain (Biotium, Inc., Fremont, Canada). Stained DNA was visualized at 302 nm.

### Purification of PCR Amplificates and Restriction Fragments

PCR amplificates and restriction fragments were cleaned up using the QIAquick PCR Purification Kit from Qiagen (Hilden, Germany; Cat. No. 28106), according to the manufacturer's instructions. DNA was eluted with 30 μl 10 mM Tris*HCl (pH 8.5).

### Determining DNA Concentration

DNA concentration was measured using the NanoDrop Spectrophotometer ND-1000 from PEQLAB Biotechnologie GmbH, since 2015 VWR brand (Erlangen, Germany).

### Assembly Cloning

Plasmid vectors were assembled using the “NEBuilder HiFi DNA Assembly Cloning Kit” purchased from New England BioLabs Inc. (Ipswich, USA, Cat. No. E5520). The reaction mix, containing the linear vector and at least one DNA insert, was incubated at 50° C. for 60 min. 0.5 μl of Assembly mixture was used for each transformation experiment.

Chemical Transformation of E. coli

For plasmid cloning, chemically competent “NEB® Stable Competent E. coli (High Efficiency)” (New England BioLabs Inc., Ipswich, USA, Cat. No. C3040) were transformed according to the manufacturer's protocol. Successfully transformed cells were selected on LB agar supplemented with 25 mg/l kanamycin.

Transformation of C. glutamicum

Transformation of C. glutamicum with plasmid-DNA was conducted via electroporation using a “Gene Pulser Xcell” (Bio-Rad Laboratories GmbH, Feldkirchen, Germany) as described by Ruan et al. (2015). Electroporation was performed in 1 mm electroporation cuvettes (Bio-Rad Laboratories GmbH, Feldkirchen, Germany) at 1.8 kV and a fixed time constant set to 5 ms. Transformed cells were selected on BHI-agar containing 134 g/l sorbitol, 2.5 g/l Yeast Extract and 25 mg/l kanamycin.

### Determining Nucleotide Sequences

Nucleotide sequences of DNA molecules were determined by Eurofins Genomics GmbH (Ebersberg, Germany) by cycle sequencing, using the dideoxy chain termination method of Sanger et al. (Proceedings of the National Academy of Sciences USA 74, 5463-5467, 1977). Clonemanager Professional 9 software from Scientific & Educational Software (Denver, USA) was used to visualize and evaluate the sequences as well as for in silico assembly of sequences.

Glycerol Stocks of E. coli and C. glutamicum Strains

For long time storage of E. coli- and C. glutamicum strains glycerol stocks were prepared. Selected E. coli clones were cultivated in 10 ml LB medium supplemented with 2 g/l glucose. Selected C. glutamicum clones were cultivated in 10 ml twofold concentrated BHI medium supplemented with 2 g/l glucose. Media for growing plasmid containing E. coli- and C. glutamicum strains were supplemented with 25 mg/l kanamycin. The medium was contained in 100 ml Erlenmeyer flasks with 3 baffles. It was inoculated with a loop of cells taken from a colony. The culture was then incubated for 18 h at 30° C. and 200 rpm. After said incubation period 1.2 ml 85% (v/v) sterile glycerol were added to the culture. The obtained glycerol containing cell suspension was then aliquoted in 2 ml portions and stored at −80° C.

### GAA Production in Millilitre-Scale Cultivations

The millilitre-scale cultivation system according to Duetz (2007) was used to assess the GAA-production of the strains. For this purpose, 24-deepwell microplates (24 well WDS plates) from EnzyScreen BV (Heemstede, Netherlands, Cat. no. CR1424) filled with 2.5 ml medium per well were used.

Precultures of the strains were done in 10 ml seed medium (SM). The medium was contained in a 100 ml Erlenmeyer flask with 3 baffles. It was inoculated with 100 μl of a glycerol stock culture and the culture was incubated for 24 h at 30° C. and 200 rpm. The composition of the seed medium (SM) is shown in Table 5.

TABLE 5
Seed medium (SM)

| Components | Concentration (g/l) |
|---|---|
| Yeast extract FM902 | 10 |
| (Angel Yeast Co., LTD, Hubei, P.R. China) | |
| Urea | 1.5 |
| KH₂PO₄ | 0.5 |
| K₂HPO₄ | 0.5 |
| MgSO₄ * 7H₂O | 1 |
| Biotin | 0.0001 |
| Thiamine hydrochloride | 0.0001 |
| FeSO₄ * 7H₂O | 0.01 |
| MnSO₄ * H₂O | 0.01 |
| Glucose | 20 |
| Kanamycin | 0.025 |
| pH = 7.0 | |

After said incubation period the optical densities OD600 of the precultures were determined. The volume, needed to inoculate 2.5 ml of production medium (PM) to an OD600 of 0.1, was sampled from the preculture, centrifuged (1 min at 8000 g) and the supernatant was discarded. Cells were then resuspended in 100 μl of production medium.

The main cultures were started by inoculating the 2.4 ml production medium (PM) containing wells of the 24 Well WDS-Plate with each 100 μl of the resuspended cells from the precultures. The composition of the production medium (PM) is shown in Table 6.

TABLE 6
Production medium (PM)

| Components | Concentration (g/l) |
|---|---|
| 3-(N-morpholino)propanesulfonic acid (MOPS) | 40 |
| Yeast extract FM902 (Angel Yeast Co., LTD, | 1.5 |
| Hubei, P.R. China) | |
| (NH₄)₂SO₄ | 10 |
| NH₄Cl | 15 |
| Trisodium citrate * 2H₂O | 10 |
| Urea | 1 |
| KH₂PO₄ | 0.5 |
| K₂HPO₄ | 0.5 |
| Ammonium acetate | 7.7 |
| MgSO₄ * 7H₂O | 1 |
| Biotin | 0.0001 |
| Thiamine hydrochloride | 0.0001 |
| FeSO₄ * 7H₂O | 0.01 |
| MnSO₄ * H₂O | 0.01 |
| ZnSO₄ * 7H₂O | 0.00002 |
| CuSO₄ * 5H₂O | 0.0004 |
| Antifoam XFO-2501 | 0.5 |
| (Ivanhoe Industries Inc., Zion, USA) | |
| Glucose | 40 |
| L-Arginine | 1.9 |
| Kanamycin | 0.025 |
| pH = 7.2 | |

The main cultures were incubated for 72 h at 30° C. and 225 rpm in an Infors HT Multitron standard incubator shaker from Infors GmbH (Bottmingen, Switzerland) until complete consumption of glucose. The glucose concentration in the suspension was analysed with the blood glucose-meter One Touch Vita® from LifeScan (Johnson & Johnson Medical GmbH, Neuss, Germany).

After cultivation the culture suspensions were transferred to a deep well microplate. A part of the culture suspension was suitably diluted to measure the OD600. Another part of the culture was centrifuged and the concentration of GAA in the supernatant was analyzed as described below.

### B) Examples

### Example 1: Cloning of the Plasmid pK18mobsacB_DargR for the Genomic Deletion of the Gene argR C. glutamicum

To raise intracellular L-Arginine formation and L-Arginine recycling from L-Ornithine, the gene argR coding for the central repressor protein ArgR controlling the L-arginine biosynthetic pathway was to be deleted. Therefore, the plasmid pK18mobsacB_DargR was constructed as follows. Plasmid pK18mobsacB (Schäfer, 1994; Genbank accession FJ437239) was digested using restriction endonuclease Xbal and the linearized vector DNA (5721 bps) was purified using the “QIAquick Gel Extraction Kit”.

For constructing the insert, two DNA fragments were created by high fidelity PCR with the following pairs of primers (using DNA of ATCC13032 as a template):
DargR_If (SEQ ID NO:1), +DargR_Ir (SEQ ID NO:2)
=left homology arm (983 bps)
DargR_rf (SEQ ID NO:3), +DargR_rr (SEQ ID NO:4)
=left homology arm (984 bps)

The PCR products were purified using the “QIAquick PCR Purification Kit”.

The linearized plasmid and the PCR products were assembled using the “NEBuilder HiFi DNA Assembly Cloning Kit”. The assembly product was transformed into “NEB Stable Competent E. coli (High Efficiency)” and cells were grown on LB agar containing 25 mg/l kanamycin. A proper plasmid clone was identified by restriction digestion and DNA sequencing. The resulting plasmid was named pK18mobsacB_DargR.

### Example 2: Chromosomal Deletion of the Gene argR in ATCC13032

C. glutamicum ATCC13032 (Kinoshita S, Udaka S, Shimono M., J. Gen. Appl. Microbiol. 1957; 3 (3): 193-205), the Corynebacterium glutamicum Type Strain/Wildtype, is commercially available at the American Type Culture Collection (ATCC) or at the DSMZ-German Collection of Microorganisms and Cell Cultures GmbH under the deposit no. DSM 20300.

For deleting the argR gene, plasmid pK18mobsacB_DargR was transformed into C. glutamicum ATCC13032 by electroporation. Chromosomal integration (resulting from a first recombination event) was selected by plating on BHI agar supplemented with 134 g/l sorbitol, 2.5 g/l yeast extract and 25 mg/l kanamycin. The agar plates were incubated for 48 h at 33° C.

Individual colonies were transferred onto fresh agar plates (with 25 mg/l kanamycin) and incubated for 24 h at 33° C. Liquid cultures of these clones were cultivated for 24 h at 33° C. in 10 ml BHI medium contained in 100 ml Erlenmeyer flasks with 3 baffles. To isolate clones that have encountered a second recombination event, an aliquot was taken from each liquid culture, suitably diluted and 100 μl were plated on BHI agar supplemented with 10% saccharose. The agar plates were incubated for 48 h at 33° C. Colonies growing on the saccharose containing agar plates were then examined for kanamycin sensitivity. To do so a toothpick was used to remove cell material from the colony and to transfer it onto BHI agar containing 25 mg/l kanamycin and onto BHI agar containing 10% saccharose. The agar plates were incubated for 60 h at 33° C. Clones that proved to be sensitive to kanamycin and resistant to saccharose were examined by PCR and DNA sequencing. The resulting strain having a deleted argR gene was named ATCC13032_DargR.

### Example 3: Cloning of the Plasmid pK18mobsacB_CK for the Genomic Integration of the Carbamate Kinase (CK, EC 2.7.2.2) Gene Derived from Enterococcus faecalis ATCC 29212 into C. glutamicum

The coding sequence arcC of Enterococcus faecalis ATCC 29212 codes for a carbamate kinase (Marina et al., Eur J Biochem. 1998 Apr. 1; 253 (1): 280-91. doi: 10.1046/j.1432-1327.1998.2530280.x; Genbank accession AJ223332, SEQ ID NO:5). SEQ ID NO:6 shows the derived amino acid sequence (Genbank accession CAA11271).

Using the software tool “Codon Optimization Tool” (Integrated DNA Technologies Inc., Coralville, Iowa, USA) the coding sequence was optimized for the codon usage of C. glutamicum. The resulting optimized coding sequence was named CK.

With the optimized coding sequence, a DNA fragment for the genomic integration in C. glutamicum between the genes NCgl0291 and NCgl0292 was designed. It consists of the following elements: a Bsal restriction site, a homologous sequence for assembly cloning into pK18mobsacB (Schäfer, 1994; Genbank accession FJ437239), a left homologous arm for integration downstream of NCg10291, the strong sod-promotor from C. glutamicum, the optimized CK gene, the BioBricks Terminator BBa_B1006, a right homologous arm for genomic integration, a second homologous sequence for assembly cloning and a Bsal site. The resulting DNA sequence was named CK-insert (SEQ ID NO:7). It was ordered for gene synthesis from Invitrogen/Geneart (Thermo Fisher Scientific, Waltham, USA) and it was delivered as part of a cloning plasmid with an ampicillin resistance gene.

The cloning plasmid containing the CK-insert was digested using the restriction endonuclease Bsal and the DNA was purified with the “QIAquick PCR Purification Kit”.

The plasmid pK18mobsacB was digested using the restriction endonuclease Smal and the DNA was purified with the “QIAquick PCR Purification Kit”.

The DNA of both digested plasmids was joined, and the matching sequence ends were assembled using the “NEBuilder HiFi DNA Assembly Cloning Kit”. The assembly product was transformed into “NEB Stable Competent E. coli (High Efficiency)” and cells were grown on LB agar containing 25 mg/l kanamycin. A proper plasmid clone was identified by restriction digestion and DNA sequencing. The resulting plasmid was named pK18mobsacB_CK.

### Example 4: Integration of the Carbamate Kinase Gene CK into the Genome of C. glutamicum

For the genomic integration of the carbamate kinase gene CK into C. glutamicum ATCC13032 and ATCC13032_DargR, the strains were transformed by plasmid pK18mobsacB_CK using electroporation. Chromosomal integration (resulting from a first recombination event) was selected by plating on BHI agar supplemented with 134 g/l sorbitol, 2.5 g/l yeast extract and 25 mg/l kanamycin. The agar plates were incubated for 48 h at 33° C.

Individual colonies were transferred onto fresh agar plates (with 25 mg/l kanamycin) and incubated for 24 h at 33° C. Liquid cultures of these clones were cultivated for 24 h at 33° C. in 10 ml BHI medium contained in 100 ml Erlenmeyer flasks with 3 baffles. To isolate clones that have encountered a second recombination event, an aliquot was taken from each liquid culture, suitably diluted and 100 μl were plated on BHI agar supplemented with 10% saccharose. The agar plates were incubated for 48 h at 33° C. The colonies growing on the saccharose containing agar plates were then examined for kanamycin sensitivity. To do so a toothpick was used to remove cell material from the colony and to transfer it onto BHI agar containing 25 mg/l kanamycin and onto BHI agar containing 10% saccharose. The agar plates were incubated for 60 h at 33° C. Clones that proved to be sensitive to kanamycin and resistant to saccharose were examined by PCR and DNA sequencing for the appropriate integration of the CK gene. The resulting strains were named ATCC13032_CK and ATCC13032_DargR_CK, respectively.

TABLE 7
List of strains

| Strain | Comment |
|---|---|
| ATCC13032 | Corynebacterium glutamicum wild type strain |
| | (Kinoshita et al., 1957*) |
| ATCC13032_DargR | Chromosomal deletion of argR in C. glutamicum ATCC13032 |
| ATCC13032_CK | Chromosomal insertion of the CK gene between |
| | NCgl0291 and NCg10292 in C. glutamicum ATCC13032 |
| ATCC13032_DargR_CK | Chromosomal deletion of argR and chromosomal |
| | insertion of the CK gene between NCgl0291 and |
| | NCgl0292 in C. glutamicum ATCC13032 |

### Example 5: Cloning of the Gene AGAT-Mp Coding for an L-Arginine: Glycine Amidinotransferase (AGAT, EC 2.1.4.1) Derived from Moorena Producens

Moorena producens is a filamentous cyanobacterium. The genome of the Moorena producens strain PAL-Aug. 15, 2008-1 was published by Leao et al. (Leao T, Castelão G, Korobeynikov A, Monroe E A, Podell S, Glukhov E, Allen E E, Gerwick W H, Gerwick L, Proc Natl Acad Sci USA. 2017 Mar. 21; 114 (12): 3198-3203. doi: 10.1073/pnas. 1618556114; Genbank accession Number CP017599.1). It contains an open reading frame coding for a L-arginine:glycine amidinotransferase (AGAT, EC 2.1.4.1; locus_tag BJP34_00300 shown in SEQ ID NO:8). SEQ ID NO:9 shows the derived amino acid sequence (Genbank accession Number WP_070390602).

Using the software tool “Optimizer” (http://genomes.urv.es/OPTIMIZER/) the amino acid sequence was translated back into a DNA sequence optimized for the codon usage of C. glutamicum. The 5′-end of the optimized gene was expanded with a Bsal restriction site, a 5′-UTR sequence for assembly cloning and a ribosomal binding site. At the 3′-end a second stop-codon, a sequence for assembly cloning and a Bsal-site was added. The resulting DNA sequence was named AGAT-Mp-insert (SEQ ID NO:10). It was ordered for gene synthesis from Eurofins Genomics GmbH (Ebersberg, Germany) and it was delivered as part of a cloning plasmid with an ampicillin resistance gene.

The cloning plasmid containing the AGAT-Mp-insert was digested using the restriction endonuclease Bsal and the DNA was purified with the “QIAquick PCR Purification Kit”.

The E. coli-C. glutamicum shuttle plasmid pLIB_P consists of the replication origin from pBL1 (for C. glutamicum), the pSC101 replication origin (for E. coli) and a kanamycin resistance gene. Following a unique NotI restriction site it has a strong promoter, two inversely orientated Bsal-sites and the BioBricks Terminator BBa_B1006 (SEQ ID NO:11).

pLIB_P was digested using the restriction endonuclease Bsal and the DNA was purified with the “QIAquick PCR Purification Kit”.

The DNA solutions of both Bsal digested plasmids were joined, and matching sequence ends were assembled using the “NEBuilder HiFi DNA Assembly Cloning Kit”. The product was transformed into “NEB Stable Competent E. coli (High Efficiency)” and cells were grown on LB agar containing 25 mg/l kanamycin. A proper plasmid clone was identified by restriction digestion and DNA sequencing. The resulting plasmid was named pLIB_P_AGAT-Mp.

### Example 6: Synthesis of the Gene AaDH-Mt Coding for an NADH Dependent AaDH Derived from Mycobacterium tuberculosis H37Ra

The open reading frame MRA_2804 of Mycobacterium tuberculosis H37Ra presumably codes for an NADH dependent amino acid dehydrogenase (Genbank accession CP000611 locus_tag=“MRA_2804”, SEQ ID NO: 12). SEQ ID NO: 13 shows the derived amino acid sequence.

Using the software tool “Codon Optimization Tool” (Integrated DNA Technologies Inc., Coralville, Iowa, USA) the open reading frame was optimized for the codon usage of C. glutamicum. The resulting sequence was expanded with a 5′-UTR consisting of a Bsal restriction site, a homologous region for assembly cloning, the strong Pg3N3 promoter and a ribosomal binding site. Additionally, the 3′-end was expanded with a random spacer sequence, a homologous region for assembly cloning and a Bsal restriciton site. The resulting DNA sequence was named AaDH-Mt-insert (SEQ ID NO: 14). It was ordered for gene synthesis from Invitrogen/Geneart (Thermo Fisher Scientific, Waltham, USA) and it was delivered as part of a cloning plasmid with an ampicillin resistance gene. The optimized gene was named AaDH-Mt.

### Example 7: Synthesis of the Gene AtGGT1 Coding for a Glyoxylate Aminotransferase Derived from Arabidopsis thaliana

The gene GGT1 of Arabidopsis thaliana (Genbank accession Number NM_102180, SEQ ID NO: 15) codes for a glutamate: glyoxylate aminotransferase (Genbank accession Number NP_564192, SEQ ID NO: 16). The protein has been shown to catalyze the reactions glyoxylate+L-alanine=glycine+pyruvate (EC 2.6.1.44), 2-oxoglutarate+L-alanine=L-glutamate+pyruvate (EC 2.6.1.2), and 2-oxoglutarate+glycine=glyoxylate+L-glutamate (EC 2.6.1.4; Liepman A H, Olsen L J., Plant Physiol. 2003 January; 131 (1): 215-27. doi: 10.1104/pp. 011460).

Using the software tool “Codon Optimization Tool” (Integrated DNA Technologies Inc., Coralville, Iowa, USA) the amino acid sequence of the GGT1 protein was translated back into a DNA sequence optimized for the codon usage of C. glutamicum. The resulting sequence was expanded with a 5′-UTR consisting of a Bsal restriction site, a homologous region for assembly cloning and a ribosomal binding site. Additionally, the 3′-end was expanded with a homologous region for assembly cloning and a Bsal restriciton site. The resulting DNA sequence was named AtGGT1-insert (SEQ ID NO:17). It was ordered for gene synthesis from Invitrogen/Geneart (Thermo Fisher Scientific, Waltham, USA) and it was delivered as part of a cloning plasmid with an ampicillin resistance gene. The optimized gene was named AtGGT1.

### Example 8: Cloning of a Plasmid for the Co-Expression of AGAT-Mp and AaDH-Mt

For the combined expression of AGAT-Mp and AaDH-Mt, the synthetic gene AaDH-Mt was cloned into plasmid pLIB_P_AGAT-Mp.

The Plasmid pLIB_P_AGAT-Mp was digested using the restriction endonuclease NotI and the DNA was purified with the “QIAquick PCR Purification Kit”. The plasmid containing the synthetic sequence AaDH-Mt-insert (SEQ ID NO: 14) was digested using the restriction endonuclease Bsal and the resulting DNA was purified with the “QIAquick PCR Purification Kit”.

A dummy DNA was designed having compatible ends for assembly cloning. It was named dummy-insert (SEQ ID NO:18) and it was ordered for gene synthesis from Invitrogen/Geneart (Thermo Fisher Scientific, Waltham, USA) as a linear double stranded DNA fragment.

The DNA of NotI digested pLIB_P_AGAT-Mp was joined with the Bsal digested plasmid containing AaDH-Mt-insert and the dummy-insert by using the “NEBuilder HiFi DNA Assembly Cloning Kit”. The product was transformed into “NEB Stable Competent E. coli (High Efficiency)” (New England Biolabs, Ipswich, USA) and cells were grown on LB agar containing 25 mg/l kanamycin. A proper plasmid clone was identified by restriction digestion and DNA sequencing. The resulting plasmid was named pLIB_AaDH-Mt_AGAT-Mp.

### Example 9: Cloning of a Plasmid for the Co-Expression of AGAT-Mp, AaDH-Mt and AtGGT1

For the combined expression of AGAT-Mp, AaDH-Mt and AtGGT1, the synthetic genes AaDH-Mt and AtGGT1 were cloned into plasmid pLIB_P_AGAT-Mp.

The Plasmid pLIB_P_AGAT-Mp was digested using the restriction endonuclease NotI and the DNA was purified with the “QIAquick PCR Purification Kit”. The plasmid containing the synthetic sequence AaDH-Mt-insert (SEQ ID NO:14) was digested using the restriction endonuclease Bsal and the resulting DNA was purified with the “QIAquick PCR Purification Kit”. The plasmid containing the synthetic sequence AtGGT1-insert (SEQ ID NO:17) was digested using the restriction endonuclease Bsal and the resulting DNA was purified with the “QIAquick PCR Purification Kit”.

The DNA of NotI digested pLIB_P_AGAT-Mp was joined with the Bsal digested plasmid containing AaDH-Mt-insert and with the Bsal digested plasmid containing AtGGT1-insert using the “NEBuilder HiFi DNA Assembly Cloning Kit”. The product was transformed into “NEB Stable Competent E. coli (High Efficiency)” and cells were grown on LB agar containing 25 mg/l kanamycin. A proper plasmid clone was identified by restriction digestion and DNA sequencing. The resulting plasmid was named pLIB_AaDH-Mt_AtGGT1_AGAT-Mp.

### Example 10: Cloning of a Plasmid for the Co-Expression of AGAT-Mp and AtGGT1

For the combined expression of AGAT-Mp and AtGGT1, the gene AaDH-Mt was deleted from the plasmid pLIB_AaDH-Mt_AtGGT1_AGAT-Mp.

The Plasmid pLIB_AaDH-Mt_AtGGT1_AGAT-Mp was digested using the restriction endonucleases Sacl and Sall and the DNA was purified with the “QIAquick PCR Purification Kit”. The Ends of the linear DNA were blunted using the Fast DNA End Repair Kit (Thermo Fisher Scientific, Waltham, USA) and the DNA was purified. The DNA was subjected to self ligation using the Rapid Ligation Kit (Thermo Fisher Scientific, Waltham, USA). The ligated product was transformed into “NEB Stable Competent E. coli (High Efficiency)” and cells were grown on LB agar containing 25 mg/l kanamycin. A proper plasmid clone was identified by restriction digestion and the resulting plasmid was named pLIB_AtGGT1_AGAT-Mp.

### Example 11: Transformation of C. glutamicum Strains with Various Plasmids

The following strains of C. glutamicum were transformed with the various plasmids by electroporation (Table 8). Plasmid containing cells were selected with 25 mg/l kanamycin.
C. glutamicum ATCC13032: commonly used wild type strain (Kinoshita et al., J. Gen. Appl. Microbiol. 1957; 3 (3): 193-205)
C. glutamicum ATCC13032_DargR: chromosomal deletion of argR in C. glutamicum ATCC13032
C. glutamicum ATCC13032_CK: chromosomal insertion of the CK gene between NCgl0291 and NCgl0292 in C. glutamicum ATCC13032
C. glutamicum ATCC13032_DargR_CK: Chromosomal deletion of argR and chromosomal insertion of the CK gene between NCgl0291 and NCgl0292 in C. glutamicum ATCC13032

TABLE 8
List of plasmid-containing C. glutamicum strains

| Plasmid | Recipient strain | Resulting strain |
|---|---|---|
| pLIB_P | ATCC13032 | ATCC13032/pLIB_P |
| pLIB_P_AGAT-Mp | ATCC13032 | ATCC13032/pLIB_P_AGAT-Mp |
| pLIB_AaDH-Mt_AtGGT1_AGAT-Mp | ATCC13032 | ATCC13032/pLIB_AaDH- |
| | | Mt_AtGGT1_AGAT-Mp |
| pLIB_AaDH-Mt_AtGGT1_AGAT-Mp | ATCC13032_DargR | ATCC13032_DargR/pLIB_AaDH- |
| | | Mt_AtGGT1_AGAT-Mp |
| pLIB_P_AGAT-Mp | ATCC13032_CK | ATCC13032_CK/pLIB_P_AGAT- |
| | | Mp |
| pLIB_AaDH-Mt_AGAT-Mp | ATCC13032_CK | ATCC13032_CK/pLIB_AaDH- |
| | | Mt_AGAT-Mp |
| pLIB_AtGGT1_AGAT-Mp | ATCC13032_CK | ATCC13032_CK/pLIB_AtGGT1_A |
| | | GAT-Mp |
| pLIB_AaDH-Mt_AtGGT1_AGAT-Mp | ATCC13032_CK | ATCC13032_CK/pLIB_AaDH- |
| | | Mt_AtGGT1_AGAT-Mp |
| pLIB_AaDH-Mt_AtGGT1_AGAT-Mp | ATCC13032_DargR_CK | ATCC13032_DargR_CK/pLIB_AaD |
| | | H-Mt_AtGGT1_AGAT-Mp |

### Example 12: Impact of the L-Arginine: Glycine Amidinotransferase Gene on GAA Production

To assess the impact of the L-arginine:glycine amidinotransferase gene (AGAT-Mp) on GAA production, strains ATCC13032/pLIB_P and ATCC13032/pLIB_P_AGAT-Mp were cultivated in the Wouter Duetz system in production medium and the resulting GAA titers were determined as described above.

TABLE 9
Impact of the expression of the L-arginine:glycine
amidinotransferase gene on GAA production

| Strain | GAA |
|---|---|
| ATCC13032/pLIB_P | not detectable |
| ATCC13032/pLIB_P_AGAT-Mp | 120 mg/l |

The cultivation of the strain having the L-arginine:glycine amidinotransferase gene resulted in GAA production, compared to the strain lacking L-arginine:glycine amidinotransferase gene (see Table 9). We conclude that the presence of the heterologous L-arginine:glycine amidinotransferase gene enables the production of GAA.

### Example 13: Impact of the Carbamate Kinase Gene on GAA Production

To assess the impact of the carbamate kinase gene on GAA production, strains ATCC13032/pLIB_P_AGAT-Mp and ATCC13032_CK/pLIB_P_AGAT-Mp were cultivated in the Wouter Duetz system in production medium and the resulting GAA titers were determined as described above.

TABLE 10
Impact of the expression of the carbamate kinase gene on
GAA production

| Strain | GAA |
|---|---|
| ATCC13032/pLIB_P_AGAT-Mp | 120 mg/l |
| ATCC13032_CK/pLIB_P_AGAT-Mp | 223 mg/l |

The cultivation of the strain having the carbamate kinase gene resulted in a higher GAA titre, compared to the strain lacking a carbamate kinase gene (see Table 10). We conclude that the presence of the carbamate kinase gene improves the production of GAA.

### Example 14: Impact of the Combined Presence of the Carbamate Kinase Gene and the Glyoxylate Aminotransferase Gene on GAA Production

To assess the impact of the combined presence of the carbamate kinase gene (CK) and the glyoxylate aminotransferase gene (AtGGT1) on GAA production, strains ATCC13032_CK/pLIB_P_AGAT-Mp and ATCC13032_CK/pLIB_AtGGT1_AGAT-Mp were cultivated in the Wouter Duetz system in production medium and the resulting GAA titers were determined as described above.

TABLE 11
Impact of the combined presence of the carbamate kinase
gene and the glyoxylate aminotransferase gene on GAA production

| Strain | GAA |
|---|---|
| ATCC13032_CK/pLIB_P_AGAT-Mp | 223 mg/l |
| ATCC13032_CK/pLIB_AtGGT1_AGAT-Mp | 337 mg/l |

The cultivation of the strain having the carbamate kinase gene and the glyoxylate aminotransferase gene resulted in a higher GAA titre, compared to the strain lacking the glyoxylate aminotransferase gene (see Table 11). We conclude that the combined presence of the carbamate kinase gene and the glyoxylate aminotransferase gene improves the production of GAA.

### Example 15: Impact of the Combined Presence of the Carbamate Kinase Gene and the NADH-Dependent Amino Acid Dehydrogenase Gene on GAA Production

To assess the impact of the combined presence of the carbamate kinase gene (CK) and the NADH-dependent amino acid dehydrogenase gene (AaDH-Mt) on GAA production, strains ATCC13032_CK/pLIB_P_AGAT-Mp and ATCC13032_CK/pLIB_AaDH-Mt_AGAT-Mp were cultivated in the Wouter Duetz system in production medium and the resulting GAA titers were determined as described above.

TABLE 12
Impact of the combined presence of the carbamate kinase gene and the
NADH-dependent amino acid dehydrogenase gene on GAA production

| Strain | GAA |
|---|---|
| ATCC13032_CK/pLIB_P_AGAT-Mp | 223 mg/l |
| ATCC13032_CK/pLIB_AaDH-Mt_AGAT-Mp | 545 mg/l |

The cultivation of the strain having the carbamate kinase gene and the NADH-dependent amino acid dehydrogenase gene resulted in a higher GAA titer, compared to the strain lacking the NADH-dependent amino acid dehydrogenase gene (see Table 12). We conclude that the combined presence of the carbamate kinase gene and the NADH-dependent amino acid dehydrogenase gene improves the production of GAA.

### Example 16: Impact of the Combined Presence of the Carbamate Kinase Gene, the NADH-Dependent Amino Acid Dehydrogenase Gene and the Glyoxylate Aminotransferase Gene on GAA Production

To assess the impact of the combined presence of the carbamate kinase gene (CK), the glyoxylate aminotransferase gene (AtGGT1) and the NADH-dependent amino acid dehydrogenase gene (AaDH-Mt) on GAA production, strains ATCC13032_CK/pLIB_AtGGT1_AGAT-Mp, ATCC13032_CK/pLIB_AaDH-Mt_AGAT-Mp and ATCC13032_CK/pLIB_AaDH-Mt_AtGGT1_AGAT-Mp were cultivated in the Wouter Duetz system in production medium and the resulting GAA titers were determined as described above.

TABLE 13
Impact of the combined presence of the carbamate kinase gene,
the NADH-dependent amino acid dehydrogenase gene and the
glyoxylate aminotransferase gene on GAA production

| Strain | GAA |
|---|---|
| ATCC13032_CK/pLIB_AtGGT1_AGAT-Mp | 337 mg/l |
| ATCC13032_CK/pLIB_AaDH-Mt_AGAT-Mp | 545 mg/l |
| ATCC13032_CK/pLIB_AaDH-Mt_AtGGT1_AGAT-Mp | 657 mg/l |

The cultivation of the strain having a combination of the carbamate kinase gene, the NADH-dependent amino acid dehydrogenase gene and the glyoxylate aminotransferase gene resulted in a higher GAA titre, compared to the strains lacking either the NADH-dependent amino acid dehydrogenase gene or the glyoxylate aminotransferase gene (see Table 13). We conclude that the combined presence of the carbamate kinase gene, the NADH-dependent amino acid dehydrogenase gene and the glyoxylate aminotransferase gene improves the production of GAA.

### Example 17: Impact of the Deletion of the argR Gene in Combination with the Presence of the Carbamate Kinase Gene, the NADH-Dependent Amino Acid Dehydrogenase Gene and the Glyoxylate Aminotransferase Gene on GAA Production

To assess the impact of the deletion of the argR gene (DargR) in combination with the presence of the carbamate kinase gene (CK), the glyoxylate aminotransferase gene (AtGGT1) and the NADH-dependent amino acid dehydrogenase gene (AaDH-Mt) on GAA production, strains ATCC13032/pLIB_AaDH-Mt_AtGGT1_AGAT-Mp, ATCC13032_DargR/pLIB_AaDH-Mt_AtGGT1_AGAT-Mp and ATCC13032_DargR_CK/pLIB_AaDH-Mt_AtGGT1_AGAT-Mp were cultivated in the Wouter Duetz system in production medium and the resulting GAA titers were determined as described above.

TABLE 14
Impact of the deletion of the argR gene in combination
with the presence of the carbamate kinase gene, the
NADH-dependent amino acid dehydrogenase gene
and the glyoxylate aminotransferase gene on GAA production

| Strain | GAA |
|---|---|
| ATCC13032/pLIB_AaDH-Mt_AtGGT1_AGAT-Mp | 377 mg/l |
| ATCC13032_DargR/pLIB_AaDH-Mt_AtGGT1_AGAT-Mp | 589 mg/l |
| ATCC13032_DargR_CK/pLIB_AaDH-Mt_AtGGT1_AGAT-Mp | 845 mg/l |

The cultivation of the strain having a deleted argR gene in combination with the presence of the glyoxylate aminotransferase gene and the NADH-dependent amino acid dehydrogenase gene resulted in a higher GAA titer, compared to the strain with a wildtype argR gene (see Table 14). The cultivation of the strain having a deleted argR gene in combination with the presence of the carbamate kinase gene, the glyoxylate aminotransferase gene and the NADH-dependent amino acid dehydrogenase gene resulted in a higher GAA titer, compared to both strains lacking the carbamate kinase gene (see Table 14).

We conclude that the deletion of the argR gene in combination with the presence of the carbamate kinase gene, the glyoxylate aminotransferase gene and the NADH-dependent amino acid dehydrogenase gene improves the production of GAA.

1. A microorganism, comprising:
at least one heterologous gene coding for a protein having a function of a L-arginine:glycine amidinotransferase; and
at least one gene coding for a protein having a function of a carbamate kinase; and
further comprising at least one gene coding for a protein having a function of a NADH-dependent amino acid dehydrogenase.

2. The microorganism of claim 1, wherein an activity of the at least one protein having the function of a carbamate kinase is increased compared with a respective activity in a wildtype microorganism.

3. The microorganism of claim 1, comprising at least one heterologous gene coding for a protein having the function of a carbamate kinase.

4. The microorganism of claim 1, wherein an activity of the protein having the function of a NADH-dependent amino acid dehydrogenase is increased compared with the respective activity in the wildtype microorganism.

5. The microorganism of claim 1, wherein the protein having the function of a NADH-dependent amino acid dehydrogenase is a heterologous protein.

6. The microorganism of claim 1, further comprising at least one gene coding for a protein having the function of a glyoxylate aminotransferase.

7. The microorganism of claim 1, wherein the microorganism has an increased ability to produce L-arginine from L-ornithine compared with an ability of the wildtype microorganism.

8. The microorganism of claim 7, wherein an expression of an argR gene coding for an arginine responsive repressor protein ArgR is attenuated compared to an expression of the argR gene coding in the wildtype microorganism or wherein the argR gene is deleted.

9. The microorganism of claim 1, wherein the microorganism belongs to a genus Corynebacterium, to a genus Bacillus, to a genus Enterobacteriaceae or to a genus Pseudomonas.

10. A method for a fermentative production of guanidino acetic acid (GAA), the method comprising:
a) cultivating the microorganism as defined in claim 1 in a medium, and
b) accumulating GAA in the medium to form a GAA containing fermentation broth.

11. The method of claim 10, further comprising isolating GAA from the GAA containing fermentation broth.

12. The microorganism as claimed in claim 1, further comprising a gene coding for an enzyme having an activity of a guanidinoacetate N-methyltransferase.

13. The microorganism of claim 12, wherein the gene coding for an enzyme having the activity of a guanidinoacetate N-methyltransferase is overexpressed.

14. A method for the fermentative production of creatine, the method comprising:
a) cultivating the microorganism according to claim 1 in a medium, and
b) accumulating creatine in the medium to form a creatine containing fermentation broth.

15. The method of claim 14, further comprising isolating creatine from the creatine containing fermentation broth.


SEQUENCE LISTING (24 sequences)

SEQ ID NO: 1 - DNA, 44 bp, synthetic construct
  source 1..44: mol_type = other DNA
tcgagctcggtacccggggatcctctggctgatgatgatctcac

SEQ ID NO: 2 - DNA, 45 bp, synthetic construct
  source 1..45: mol_type = other DNA
ctagggatagtagacacttcacaagtgtcttacctcggctggttg

SEQ ID NO: 3 - DNA, 47 bp, synthetic construct
  source 1..47: mol_type = other DNA
cttgtgaagtgtctactatccctagaattactcagcgggcgcaccac

SEQ ID NO: 4 - DNA, 46 bp, synthetic construct
  source 1..46: mol_type = other DNA
agcttgcatgcctgcaggtcgactctgcacgacggttcagctcttc

SEQ ID NO: 5 - DNA, 933 bp, Enterococcus faecalis
  source 1..933: mol_type = genomic DNA; strain = Enterococcus faecalis ATCC 29212
atggggaaaaaaatggtcgttgcactaggcggaaatgccatcctttcaaatgatgctagcgcacacgcacaacagcaagcattagttcaaacatctgcctatttagttcatttaattaaacaagggcatcggttgattgtttcacatggcaatggtccacaagtcggaaatctattattgcaacaacaagcagctgattctgaaaaaaatccagcgatgccgctagatacgtgtgttgctatgacacaaggaagtatcggctactggttgtccaatgcgcttaatcaagaattaaacaaagcaggaatcaagaaacaagtggctactgttttaacacaggtggtcgtagatccagcagatgaggcattcaaaaatccaacaaaaccgatcggtccatttttaacagaagctgaagccaaagaagcaatgcaagcaggtgctatttttaaagaagatgcaggacgtggctggcgcaaagtcgttccaagtcctaagccaattgacatccacgaggctgagactattaataccttaataaaaaatgatataattaccatttcatgtggtggtggtggcatccctgtcgtaggacaagaattaaaaggtgtcgaagcagtaatcgataaagattttgcttctgaaaaactggcagaattagtggatgcggatgcactagttattttaactggcgtggattatgtgtgtattaattatggcaaaccagatgaaaaacaattaaccaatgtcactgtagcagagttggaagaatacaaacaagcgggtcattttgcaccaggtagtatgttaccgaaaattgaagccgccattcagtttgtagaaagccaacccaataaacaagcaattatcacttctttggaaaacttaggaagtatgagcggcgatgaaatcgtaggaacggttgtgacaaaataa

SEQ ID NO: 6 - protein, 310 aa, Enterococcus faecalis
  source 1..310: note = strain: Enterococcus faecalis ATCC 29212
MGKKMVVALGGNAILSNDASAHAQQQALVQTSAYLVHLIKQGHRLIVSHGNGPQVGNLLLQQQAADSEKNPAMPLDTCVAMTQGSIGYWLSNALNQELNKAGIKKQVATVLTQVVVDPADEAFKNPTKPIGPFLTEAEAKEAMQAGAIFKEDAGRGWRKVVPSPKPIDIHEAETINTLIKNDIITISCGGGGIPVVGQELKGVEAVIDKDFASEKLAELVDADALVILTGVDYVCINYGKPDEKQLTNVTVAELEEYKQAGHFAPGSMLPKIEAAIQFVESQPNKQAIITSLENLGSMSGDEIVGTVVTK

SEQ ID NO: 7 - DNA, 3842 bp, synthetic construct
  source 1..3842: mol_type = other DNA
caactcggtctcgacatgattacgaattcgagctcggtacccgcacaatcgcgtcgattgaacgctcccccggcatcattgccctagacggaccgttcacccacgatcacgtctccgtacgtggcattcgcctccatttagcagaggcaggctcccccaccaaacccctggttcttctgatccacggggctttcggcggttggtacgactaccgcgaagtcatcggcccactcgcagatgccggcttccacgtcgccgccatcgatctacgcggctacggcatgtccgacaaacccccaacaggctacgacctccgccacgcagccggagaactcagcagcgttatcgcagctctcggccacgatgacgcacttcttgtcggctccgacaccggcgccagcatcgcctgggctatcgcttccatgtaccccgaacgggtccgcggcctaatttccctcggcgcgatccacccccttgacatgcgacgcgccatccgacgaaaaccccacctacacgtctctgacctcagccgacttgctccttttcggttgccctcattcctgcataacctcttccacttcggaatcaccagcgaagctcgacgtgagatcgtcaacaacacgtcctcgtcctaccagcgcagcaacgcattcacagagacagtgctcctccgcaaaaaagcactatcgatcgaccacaccatcaccccgatcatccgcaccaaccgctacctcgttgggtcgatccccagcaaaacagtctccgcaccggtgtggctgctcagaaccaacactcgacgctgggaacatctagccaatactgcgcgcactcgaacgacagggccattcaccaccatcgcgatccccggcggctacgaactcccctacctcgagaacccttccgaatttgcagcaaccatcgcagagttcgcgcgcaccacgttttaagcactgtggctgaggcgctgctgctcatttggcgtcagaaggtcgcatgattttggcgtgaattagtggtttttccctggttttaccccggcgcattgaccggaccagacaggcgtgacaagaatcaagattttcgccaggttttgtcacgtgtgtctggtttgagcgactcgaaaccaaacaggcgtgccaaaacttagatgttttagcaatttttgtcacgtgtgtctggtttcatctagttcgaccgcaaacctcacggatttccccctagtcactcaaaaaccaaaactccctatatgcccctctaagcgcttgggattccccgatttccttttaggtggaatgtaaaaaagaaaccccctgaaattcaatgaagaatttcagggggtttccgctatcggctagctaagtgaattacttggttacaacggttcccacgatttcgtcgccggacatggagccgaggttttccagggaggtgatgatggcctgtttgtttggctgggactccacgaactggatagcggcttcgatctttgggagcatgctgcctggtgcgaagtggccagcctgcttgtactcctcgagttctgccacggtaacgttggtgagttgcttctcgtcaggcttgccgtagttgatgcacacataatcaacgccggtcaggatcacgagtgcgtctgcgtcaactagctctgcgagcttttcggaagcaaagtccttatcaatcacggcttccacgcccttgagttcttggccaacgactggaataccgccaccgccgcatgagatagtgatgatgtcgttcttgatcagggtgttaatagtctctgcttcgtggatgtcgataggctttggagacgggacaaccttacgccagccgcggcctgcgtcctccttgaagattgcgcccgcctgcattgcctcttttgcctctgcttcagtgaggaatgggccgattggcttggttgggttcttgaacgcttcatcggctgggtcaacgacaacctgggtcagaacggttgcgacctgctttttgataccagccttgttaagttcttggttgagagcgttggacagccagtatccgatggatccctgggtcatagcaacgcaggtgtccagtggcattgccgggttcttttcgctgtctgctgcttgctgttggagcagcaggtttccaacctgtgggccgttgccgtgggatacgatcagacggtgaccctgcttgatgaggtgaaccaggtatgcggaggtctggaccagtgcttgctgctgtgcgtgtgcggatgcgtcgttggataggattgcgttaccgcccagcgcgaccaccattttcttgcccatgggtaaaaaatcctttcgtaggtttccgcaccgagcatatacatcttttgaaaatccgtcagatggcgcttcgcaaaagtacttggtgcgacacctcccaatgataggcctttttgttgatattgcaacgaaatttttcagccgacctatttatcgggtagcgggtcacaagcccggaataattggcagctaagtagggttgaagggcataaggcttcctcaattttcgaaaggaacattcctgttatggtaccccgaaaaaaaaaccccgccctgtcaggggcggggtttttttttcccataccaatagacacctctcctattccaggcccttaaaacgccacacaggattggtcgtatctatctcggattgggcgattcactgccaagaccaaaccacactgcccacgcaacggaaaaaccgcaatcgtgggcatctgtgaccggttccgagcccccaaaaccaaaccacactgcccacgcaacggaaaaaccgcaatcgtgggcatccctgtctggtcctagctcccgacgactaagaaaccgcgcactgcatcgtatcgacaggctgagtcagcgcggtgatgtcgccgatccgctcctgtacctcttcggcagtgagaacgtaaccggtatcggagccgtcgatcgctgcgccgaagacaacaccaaccacttcacccatttcgttggtcattgggccgccggagtttccagattggatggatccgcggactgaataggcttcgcgttcgtgctggccgttggcgtaaatgttgctgccggtgatcatgatgcgttcgcggaccctggctggggaggcgttgaaaggtccggactgtgggaatcccatgacgattgcttcatcgccagtgtctagcggagtggatgcccacggcagtggatccaagccgaggtcagggctgtaaaggactgcgatgtccaggttcgggtcgtagaacactacctctgcggagcgggttccgatcatggtatccaggctgacggtggaggtacctgcaacaacgtgggcgttggtcacaacgtagtcgggggatgccacaaagccagaacccatgagtcggcggctgcattcttgggcgtcacccatcacgtggatgacggacgggcgcattgcttcgactaggtcaacgttggtgacgttgatttcgggggcgtccacttccaccgaggatccgccggtgaagggggaaatcagtggtgggaggccggattcgctgagcatcgcagcgattttggagggcagggtatctaggccttgcggggtgtatttgtctacaaagcccaggatgcgggagtctctaattccgctggcgacagttccggggaggcctgtggccaggggaattgcgacgagccacaccacgatcaaggtggccaatacttggaaaatggcgccgagcccagaatctaaggtcctggaacttcggaatttgatgttgtctctaatcgcagcacccaagtgggcgccgatgagatttcggggatcctctagagtcgacctgcaggcatgagacccattga

SEQ ID NO: 8 - DNA, 1146 bp, Moorena producens
  source 1..1146: mol_type = genomic DNA; strain = Moorena producens PAL-8-15-08-1
atgtcggaaaaaattgttaattcctggaatgaatgggatgaattggaagaaatggtggtaggaattgcagactatgctagctttgaaccaaaagaaccagggaatcatccgaaattaagaaatcaaaatttagcggaaatcattcctttccccagtggacctaaagaccctaaagtccttgaaaaagctaatgaagaattaaatggactggcttatttattaaaagaccacgatgtgatagtaagaagacccgaaaaaattgattttactaaatctctaaaaacaccttactttgaagttgcaaatcaatactgtggagtctgtcctcgggatgtcatgattacctttgggaatgaaatcatggaagcgactatgtcgaagagagctagattttttgaatacttaccttaccggaaattggtctatgaatattggaataaagacgagcatatgatttggaatgctgcgcctaaaccgactatgcaggatagtatgtatctagagaatttctgggagctgtctttagaagaacgatttaagcgtatgcatgattttgaattttgtattacacaagatgaagtaatttttgatgcggctgactgtagcagattaggaaaggatatattagttcaggaatcgatgacaacaaatagaacaggaattcggtggttaaaaaagcacctagaaccaagaggatttcgggttcaccctgttcattttccccttgattttttcccctcacacattgactgtacgtttgttcctttgcgaccaggtcttattttgacaaaccctgaaagacctatacgggaagaggaggagaagatttttaaagagaatggctgggagttgatcacagttcctcaaccgacttgctcgaatgatgaaatgccaatgttttgccagtccagtaagtggttgtcaatgaatgttctgagtatatcaccgacaaaggttatctgtgaggaaagagaaaaacctctccaagaattgttggataagcatggatttgaggtttttcctttaccctttagacatgtctttgaatttggggggtcttttcattgtgcaacttgggatattcgccgaaaaggtgagtgtgaagattatttaccaaatttaaactatcaaccgatttgtggttaa

SEQ ID NO: 9 - protein, 381 aa, Moorena producens
  source 1..381: note = strain: Moorena producens PAL-8-15-08-1
MSEKIVNSWNEWDELEEMVVGIADYASFEPKEPGNHPKLRNQNLAEIIPFPSGPKDPKVLEKANEELNGLAYLLKDHDVIVRRPEKIDFTKSLKTPYFEVANQYCGVCPRDVMITFGNEIMEATMSKRARFFEYLPYRKLVYEYWNKDEHMIWNAAPKPTMQDSMYLENFWELSLEERFKRMHDFEFCITQDEVIFDAADCSRLGKDILVQESMTTNRTGIRWLKKHLEPRGFRVHPVHFPLDFFPSHIDCTFVPLRPGLILTNPERPIREEEEKIFKENGWELITVPQPTCSNDEMPMFCQSSKWLSMNVLSISPTKVICEEREKPLQELLDKHGFEVFPLPFRHVFEFGGSFHCATWDIRRKGECEDYLPNLNYQPICG

SEQ ID NO: 10 - DNA, 1362 bp, synthetic construct
  source 1..1362: mol_type = other DNA
ggtctccatccgcgtcggagacgaagtaattgacggcagcacctcgggcaaactcgagcgtctgcgggcaagcttcgcataaagacacgacgaattagacaacattagtaatgctggaagaaacaaccgagagcaggaagaacatgtcagaaaagattgttaactcctggaacgaatgggacgaacttgaagaaatggtggttggcatcgcggactacgcttccttcgagccaaaagaaccaggtaaccaccctaagctacgcaaccagaacctggccgagatcatcccattcccatccggcccaaaggacccaaaggttctcgaaaaagcaaacgaagagctgaacggcctcgcatacctcctaaaggaccacgatgttatcgtccgccgcccagaaaaaattgatttcactaagtcccttaaaaccccatacttcgaggtcgctaaccagtactgcggcgtttgcccacgcgacgttatgattaccttcggtaatgagattatggaggctaccatgagcaagcgtgcacgcttcttcgagtacttgccataccgtaagctcgtctacgagtactggaacaaggatgagcatatgatctggaacgctgctccaaagcctacgatgcaggactccatgtacttggagaacttctgggagctgtccctcgaggagcgcttcaagcgcatgcacgacttcgaattttgcattacccaggacgaggttatcttcgacgcagctgactgctcccgcctgggcaaggacatcctggtccaggagtccatgaccaccaaccgcaccggcattcgttggttgaagaagcacctcgaacctcgcggtttccgcgttcacccagtccacttccctctcgacttcttcccttctcacattgactgcactttcgtccctctccgtcccggcctcatccttaccaacccagagcgcccaatccgcgaagaagaagaaaagatcttcaaggagaacggctgggagcttatcaccgtcccacaacctacctgctccaacgacgaaatgcctatgttctgccagtcctcgaagtggctcagcatgaacgtcctttctatctccccaacaaaggttatttgcgaagagcgtgaaaagccactacaggaactcctggacaagcacggcttcgaagtcttcccactcccattccgccacgttttcgaattcggcggctcctttcactgcgccacttgggacatccgccgcaagggcgaatgcgaagactacctcccaaacctgaactaccagcctatctgcggctaataagctcggtacccggggatccgacgtcgcgttaacttaaggtaccgaattctaagcttcacaaaaagagacc

SEQ ID NO: 11 - DNA, 7061 bp, synthetic construct
  source 1..7061: mol_type = other DNA
gacagtaagacgggtaagcctgttgatgataccgctgccttactgggtgcattagccagtctgaatgacctgtcacgggataatccgaagtggtcagactggaaaatcagagggcaggaactgcagaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgccattcgccattcaggctgcctgcagggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatgggcctgcaggcctcagcgcgattcaggcctggtatgagtcagcaacaccttcttcacgaggcagatctcagcgcccccccccccctagcttgtctacgtctgatgctttgaatcggacggacttgccgatcttgtatgcggtgatttttccctcgtttgcccactttttaatggtggccggggtgagagctacgcgggcggcgacctgctgcgctgtgatccaatattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagttcctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctagattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttgacaatggctgatttcatcaggaatggaactgtcatgctgttatgtgcctggctcctaatcaaagctggggacaatgggttgccccgttgatctgatctagttcggattggcggggcttcactgtatctgggggtggcatcgtgaatagattgcacaccgtagtgggcagtgtgcacaccatagtggccatgagcaccaccacccccagggacgccgacggcgcgaagctctgcgcctggtgcggctcggagatcaagcaatccggcgtcggccggagccgggactactgccgccgctcctgccgccagcgggcgtacgaggcccggcgccagcgcgaggcgatcgtgtccgccgtggcgtcggcagtcgctcgccgagatacgtcacgtgacgaaatgcagcagccttccattccgtcacgtgacgaaactcgggccgcaggtcagagcacggttccgcccgctccggccctgccggacccccggcatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatccgagtccaatgtgtccgtgttcacctcagcggccgcaccggtcggggtaccttgacaaatactttccaatggggtacaatggcgcagtgccatccactccgaggttgataccagcctcctcggtggaatgatcatccgcgtcggagacgaagtaattgacggcagcacctcggggagacctcaaatggtctcgcgttaacttaaggtaccgaattctaagcttcacaaaaaaaaaccccgcccctgacagggcggggttttttttactcagcatgtggatggccagtggtcggaccgagcgctttaattaattagcatggtgacacaagcacagtat

SEQ ID NO: 12 - DNA, 1116 bp, Mycobacterium tuberculosis
  source 1..1116: mol_type = genomic DNA; strain = Mycobacterium tuberculosis H37Ra
atgcgcgtcggtattccgaccgagaccaaaaacaacgaattccgggtggccatcaccccggccggcgtcgcggaactaacccgtcgtggccatgaggtgctcatccaggcaggtgccggagagggctcggctatcaccgacgcggatttcaaggcggcaggcgcgcaactggtcggcaccgccgaccaggtgtgggccgacgctgatttattgctcaaggtcaaagaaccgatagcggcggaatacggccgcctgcgacacgggcagatcttgttcacgttcttgcatttggccgcgtcacgtgcttgcaccgatgcgttgttggattccggcaccacgtcaattgcctacgagaccgtccagaccgccgacggcgcactacccctgcttgccccgatgagcgaagtcgccggtcgactcgccgcccaggttggcgcttaccacctgatgcgaacccaagggggccgcggtgtgctgatgggcggggtgcccggcgtcgaaccggccgacgtcgtggtgatcggcgccggcaccgccggctacaacgcagcccgcatcgccaacggcatgggcgcgaccgttacggttctagacatcaacatcgacaaacttcggcaactcgacgccgagttctgcggccggatccacactcgctactcatcggcctacgagctcgagggtgccgtcaaacgtgccgacctggtgattggggccgtcctggtgccaggcgccaaggcacccaaattagtctcgaattcacttgtcgcgcatatgaaaccaggtgcggtactggtggatatagccatcgaccagggcggctgtttcgaaggctcacgaccgaccacctacgaccacccgacgttcgccgtgcacgacacgctgttttactgcgtggcgaacatgcccgcctcggtgccgaagacgtcgacctacgcgctgaccaacgcgacgatgccgtatgtgctcgagcttgccgaccatggctggcgggcggcgtgccggtcgaatccggcactagccaaaggtctttcgacgcacgaaggggcgttactgtccgaacgggtggccaccgacctgggggtgccgttcaccgagcccgccagcgtgctggcctga

SEQ ID NO: 13 - protein, 371 aa, Mycobacterium tuberculosis
  source 1..371: note = strain: Mycobacterium tuberculosis H37Ra
MRVGIPTETKNNEFRVAITPAGVAELTRRGHEVLIQAGAGEGSAITDADFKAAGAQLVGTADQVWADADLLLKVKEPIAAEYGRLRHGQILFTFLHLAASRACTDALLDSGTTSIAYETVQTADGALPLLAPMSEVAGRLAAQVGAYHLMRTQGGRGVLMGGVPGVEPADVVVIGAGTAGYNAARIANGMGATVTVLDINIDKLRQLDAEFCGRIHTRYSSAYELEGAVKRADLVIGAVLVPGAKAPKLVSNSLVAHMKPGAVLVDIAIDQGGCFEGSRPTTYDHPTFAVHDTLFYCVANMPASVPKTSTYALTNATMPYVLELADHGWRAACRSNPALAKGLSTHEGALLSERVATDLGVPFTEPASVLA

SEQ ID NO: 14 - DNA, 1700 bp, synthetic construct
  source 1..1700: mol_type = other DNA
caactcggtctctatccgagtccaatgtgtccgtgttcacctcagcggccattgaagcctaaaaacgaccgagcctattgggattaccattgaagccagtgtgagttgcatcacattggcttcaaatctgagactttaatttgtggattcacgggggtgtaatgtagttcataattaaccccattcgggggagcagatcgtagtgcgaacgatttcaggttcgttccctgcaaaaactatttagcgcaagtgttggaaatgcccccgtttggggtcaatgtccatttttgaatgtgtctgtatgattttgcatctgttgccaaatctttgtttccccgctataattgaggacaggttgacacggagttgactcgacgaattatccaatgtgagtaggtttggtgcgtgagttgaaaaaattcgccatactcgcccttgggttctgtcagctcaagaattcttgagtgaccgatgctctgattgacctaactgcttgacacattgcatttcctacaatcgcgagaggagacacaacatgcgcgtgggtatcccaaccgaaaccaagaacaacgagttccgcgtcgcaatcaccccagctggtgtcgctgagctcacccgccgtggccacgaagtactgatccaggcaggcgccggtgagggctccgccatcaccgatgctgacttcaaagcagccggcgctcaactggtcggcaccgctgaccaggtgtgggccgacgcagacctgctgctgaaggtcaaagaacctattgccgcagaatacggccgcctgcgccacggccagatcctgttcaccttcctccacctggcagcttcccgtgcgtgcaccgacgctctgctggactctggcaccacctcgatcgcttacgagactgtacagactgctgatggtgcactcccacttctcgccccaatgtccgaggtggcaggccgcctcgcagcacaagttggcgcataccacctgatgcgtacccagggcggtcgcggagtcctcatgggcggagtccctggcgtcgaaccagctgacgtcgtagtcatcggagccggcaccgcaggctacaacgccgctcgtatcgcaaacggcatgggtgcaaccgtcaccgtcctggacattaatatcgacaagcttcgccagctcgacgctgaattctgcggtcgtattcacacccgctactcctctgcatatgaacttgagggcgctgtcaagcgtgctgacctggttatcggcgcagtcctggttccaggcgctaaggcaccaaagctcgtgtccaactccctggtcgcacacatgaagcctggcgcagtcctggtcgacatcgctatcgaccaaggcggatgcttcgagggctcccgcccaaccacctacgaccacccaaccttcgccgtgcacgacaccctcttctactgtgttgcaaacatgccagcatccgttccaaagacctccacatacgctctcaccaacgcaaccatgccatacgttctggaactggctgaccacggctggcgcgctgcatgtcgctccaaccctgccctggcgaagggtttgagtacccacgagggtgcactcctgtcagaacgtgtcgcaaccgacttgggcgttcccttcaccgaaccagcctccgtgctcgcttaataagctcggtacccggggatccgacgtcaggaaaggagagacccattga

SEQ ID NO: 15 - DNA, 1446 bp, Arabidopsis thaliana
  source 1..1446: mol_type = genomic DNA
atggctctcaaggcattagactacgatactctgaatgaaaacgtcaagaagtgtcagtatgccgtaagaggtgaactttatctccgagcttctgagctgcagaaagaaggcaaaaagattattttcacaaacgttgggaaccctcatgctttaggacagaagccattgacatttcctcgccaggtggttgcgctttgccaagctccgtttctactagatgacccaaatgttggaatgctatttccagctgatgctattgcaagagctaaacattatctttccttgacttcaggcggtttaggtgcttacagtgattcaagaggccttccaggagttaggaaagaggttgctgagttcattcaacggcgtgatgggtatccaagtgacccagaactcatctttctcactgatggagctagcaaaggtgtgatgcaaatcttgaattgtgttatacgcggtaatggagatgggattctagttccggttccacagtatccactttactcagctaccatatcactgttaggtggtactcttgttccttactatcttgatgagtctgaaaactggggacttgatgttgctaaccttcgacaatccgttgctcaggctcgttctcaagggataacagtaagggcaatggtgatcattaaccctgggaacccaactggccagtgtctaagcgaagctaacataagagagatattgaagttctgttataacgagaaactggttcttctgggagacgaggtttatcagcagaacatataccaggatgagcgtccctttatcagctccaagaaggttttgatggaaatgggttcgccgttcagcaaggaagttcagcttgtatcttttcacacagtctctaaaggatattggggtgaatgtggacagcgaggtggatactttgagatgaccaacctccctccaagggttgttgaggagatatacaaggttgcatcaattgccctcagccctaatgtctctgcgcaaatctttatgggtttgatggttaatcctccaaagcctggagacatttcatatgaccagttcgcccgtgaaagcaaggggattcttgaatctttgagaagaagagcaaggctcatgacagatggattcaacagctgcaaaaacgtcgtgtgcaatttcacagaaggtgcaatgtattcgtttcctcaaatacggttaccaacgggagctctccaagctgcaaaacaagctggaaaagtgccagacgttttctactgtctcaagctcttagaagccacaggaatctccacagtacctggctctggatttggacagaaagaaggtgtgttccatctgaggacaacaatcctgccagcagaagatgagatgccggagatcatggatagcttcaagaagttcaacgacgagttcatgactcagtatgataataactttggttattcgaaaatgtga

SEQ ID NO: 16 - protein, 481 aa, Arabidopsis thaliana
MALKALDYDTLNENVKKCQYAVRGELYLRASELQKEGKKIIFTNVGNPHALGQKPLTFPRQVVALCQAPFLLDDPNVGMLFPADAIARAKHYLSLTSGGLGAYSDSRGLPGVRKEVAEFIQRRDGYPSDPELIFLTDGASKGVMQILNCVIRGNGDGILVPVPQYPLYSATISLLGGTLVPYYLDESENWGLDVANLRQSVAQARSQGITVRAMVIINPGNPTGQCLSEANIREILKFCYNEKLVLLGDEVYQQNIYQDERPFISSKKVLMEMGSPFSKEVQLVSFHTVSKGYWGECGQRGGYFEMTNLPPRVVEEIYKVASIALSPNVSAQIFMGLMVNPPKPGDISYDQFARESKGILESLRRRARLMTDGFNSCKNVVCNFTEGAMYSFPQIRLPTGALQAAKQAGKVPDVFYCLKLLEATGISTVPGSGFGQKEGVFHLRTTILPAEDEMPEIMDSFKKFNDEFMTQYDNNFGYSKM

SEQ ID NO: 17 - DNA, 1548 bp, synthetic construct
  source 1..1548: mol_type = other DNA
caactcggtctcgctcggtacccggggatccgacgtcaggaaaggagaggattgatggctctgaaggctctcgattacgacaccctgaacgagaacgtcaagaagtgccagtacgctgtgcgcggcgagctgtaccttcgtgcttccgagctgcagaaggagggcaagaagattattttcaccaacgtaggcaaccctcacgctctgggccagaagccacttaccttcccacgccaggttgttgctctttgccaggcaccattcctgctggatgatcctaacgtaggtatgctgttcccagctgacgctattgctcgcgctaagcactacctgtctctgacttccggtggtcttggcgcttactccgattcccgcggcctgccaggtgtccgcaaggaggtggctgagttcattcagcgccgcgacggctacccatccgaccctgaactcatcttccttaccgatggtgcttctaagggtgtaatgcagattctcaactgtgtgattcgcggtaacggcgatggtatccttgtcccagtcccacagtacccactgtactccgctactatttctcttctcggcggcaccctggttccatactacctggacgaatccgagaactggggcctcgacgtagctaaccttcgtcagtccgtcgcacaggctcgttcccagggcatcactgtccgcgctatggttattattaacccaggcaacccaactggccagtgcctgtccgaagcaaacattcgtgagatccttaagttctgctacaacgagaagctggtactcttaggcgacgaggtataccagcagaacatttaccaggatgaacgccctttcatctcctccaagaaggtactgatggagatgggctcgccattctccaaggaagttcagctggtttccttccacactgtctctaagggttactggggtgaatgtggccagcgcggcggctacttcgaaatgactaacctcccacctcgcgtcgtggaagagatctacaaggttgcatctattgctctgtccccaaacgtatccgctcagatcttcatgggcctgatggtaaacccacctaagcctggcgacatttcctacgaccagttcgctcgtgaatctaagggtatccttgaatcccttcgtcgccgcgctcgtctgatgactgacggcttcaactcctgtaagaacgtagtatgcaacttcaccgaaggcgctatgtactctttcccacagatccgtcttccaaccggtgcactccaggctgctaagcaggctggcaaggtgccagatgtgttctactgtctcaagctgctggaggctaccggcatctccactgttccaggctccggcttcggccagaaggagggcgttttccacctgcgtaccactatccttcctgctgaggatgagatgcctgaaattatggattctttcaagaagttcaacgacgagttcatgactcagtacgacaacaacttcggctactccaagatgtaataaggccgcaccggtcggggtaccttgacaaatactgagacccattga

SEQ ID NO: 18 - DNA, 156 bp, synthetic construct
  source 1..156: mol_type = other DNA
gctcggtacccggggatccgacgtcaggaaaggagaggattgatgaattcaggtatctcggagatcgcgagagctcgtaactgcgacggacacctaatgccctttagtaccaaatgggggagttaataaggccgcaccggtcggggtaccttgaca

SEQ ID NO: 19 - protein, 481 aa, Arabidopsis thaliana
MSLKALDYESLNENVKNCQYAVRGELYLRASELQKEGKKIIFTNVGNPHALGQKPLTFPRQVVSLCQAPFLLDDPNVGMIFPADAIARAKHYLSLTSGGLGAYSDSRGLPGVRKEVAEFIERRDGYPSDPELIFLTDGASKGVMQILNCVIRGQKDGILVPVPQYPLYSATISLLGGTLVPYYLEESENWGLDVNNLRQSVAQARSQGITVRAMVIINPGNPTGQCLSEANIREILRFCCDERLVLLGDEVYQQNIYQDERPFISSKKVLMDMGAPISKEVQLISFHTVSKGYWGECGQRGGYFEMTNIPPRTVEEIYKVASIALSPNVSAQIFMGLMVSPPKPGDISYDQFVRESKGILESLRRRARMMTDGFNSCKNVVCNFTEGAMYSFPQIKLPSKAIQAAKQAGKVPDVFYCLKLLEATGISTVPGSGFGQKEGVFHLRTTILPAEEEMPEIMDSFKKFNDEFMSQYADNFGYSRM

SEQ ID NO: 20 - protein, 407 aa, Thermococcus litoralis
MDYTKYLAGRANWIKGSALADVMKKASELQKKGVKLISLAAGDPDPELIPRAVLGEIAKEVLEKEPKSVMYTPANGIPELREELAAFLKKYDHLEVSPENIVITIGGTGALDLLGRVLIDPGDVVITENPSYINTLLAFEQLGAKIEGVPVDNDGMRVDLLEEKIKELKAKGQKVKLIYTIPTGQNPMGVTMSMERRKALLEIASKYDLLIIEDTAYNFMRYEGGDIVPLKALDNEGRVIVAGTLSKVLGTGFRIGWIIAEGEILKKVLMQKQPIDFCAPAISQYIALEYLKRGYFEKYHLEGALLGYKEKRDIMLKALENHLPNAEFTKPIAGMFVMFFLPEGADGISFANELMEREGVVVVPGKPFYTDESGKNAIRLNFSRPSKEEIPIGIKKLAKLYKEKFGE

SEQ ID NO: 21 - protein, 371 aa, Mycolicibacterium smegmatis
  source 1..371: note = strain: Mycolicibacterium smegmatis MC2 155
MLVGIPTEIKNNEYRVAITPAGVAELTRRGHEVIIQAGAGEGSAISDRDFKAAGAEIVNTADQVWSEAELLLKVKEPIEPEYSRMRKGQTLFTYLHLAASKPCTDALLASGTTSIAYETVQTAEGALPLLAPMSEVAGRLSAQVGAYHLMRSYGGRGVLMGGVPGVAPAEVVVIGAGTAGYNAARVAAGMGAHVTVFDLNINTLRRVDGEFGGRIETRYSSSLELEEAVKKADLVIGAVLVPGAKAPKLVTNSTVAHMKPGAVLVDIAIDQGGCFEDSRPTTHDEPTFKVHDTIFYCVANMPGAVPRTSTFALTNSTMPYVLKLADKGWQAACASDSALAKGLSTHDGKLLSEAVAKDLDLPFTDAAQFLA

SEQ ID NO: 22 - protein, 378 aa, Bacillus subtilis
  source 1..378: note = subspecies and strain: Bacillus subtilis subsp. subtilis str. 168
MIIGVPKEIKNNENRVALTPGGVSQLISNGHRVLVETGAGLGSGFENEAYESAGAEIIADPKQVWDAEMVMKVKEPLPEEYVYFRKGLVLFTYLHLAAEPELAQALKDKGVTAIAYETVSEGRTLPLLTPMSEVAGRMAAQIGAQFLEKPKGGKGILLAGVPGVSRGKVTIIGGGVVGTNAAKMAVGLGADVTIIDLNADRLRQLDDIFGHQIKTLISNPVNIADAVAEADLLICAVLIPGAKAPTLVTEEMVKQMKPGSVIVDVAIDQGGIVETVDHITTHDQPTYEKHGVVHYAVANMPGAVPRTSTIALTNVTVPYALQIANKGAVKALADNTALRAGLNTANGHVTYEAVARDLGYEYVPAEKALQDESSVAGA

SEQ ID NO: 23 - protein, 371 aa, Streptomyces fradiae
  source 1..371: note = strain: Streptomyces fradiae ATCC 10745 = DSM 40063
MKVGIPREVKNNEFRVAITPAGVHELVRNGHQVFVEQNAGVGSSITDDEYVAAGARILPTADEVWATADLLLKVKEPIAEEYHRLRKDQTLFTYLHLAASRECTDALLASGTTAIAYETVETANRALPLLAPMSEVAGRLAPQVGAYHLMRANGGRGVLPGGVPGVVPAKAVVIGGGVSGWNATQIAVGMGFDVTLLDRDINKLREADKIFGTKVKAVMSNSFELEKAVLDADLVIGAVLIPGAKAPKLVTNELVSRMKPGSVLVDIAIDQGGCFEDSRPTTHADPSFPVHESIFYCVANMPGAVPNTSTYALTNATLPYIVSLANNGWVEALRRDPALAKGLNTHDGKVVYRQVAEAHGLDHVELASLIG

SEQ ID NO: 24 - protein, 360 aa, Aphanothece halophytica
  source 1..360: note = strain: Aphanothece halophytica CM1
MEIGVPKETKDQEYRVGLSPSSVRSLCERKHPVFVETQAGVGAGFSDQDYEAAGATVVPDPKSAWNRELVVKVKEPLPDEYTYLQKNQVLFTYLHLAANRRLTEALIDSGISAIAYETVELPNGKLPLLNPMSIIAGRLSVQFGARYLEKQQGGRGVLLGGFPGVRSGRVVILGGGVVGTEAARMAVGIGAQVQILDVNVERLAELENLFGSRVELLYSNSSQIEAVVPEADLLIGAVLTTGKRAPKLVTRELVKQMRAGSVIVDVAVDQGGCVETLQVTSHSHPTYTEEGVVHFGVPNMPGAVPWTATQALNNSTLPYVIQLADQGLTALESNPILGKGLNVQKQQLIHPAVKEVFPDL